(19) United States
(12) Patent Application Publication
(10) Pub. No.: US 2004/0158289 A1
(43) Pub. Date: Aug. 12, 2004

(54) Method and apparatus for cell and electrical therapy of living tissue

(76) Inventors: Steven D. Girouard, Woodbury, MN (US); Rodney W. Salo, Fridley, MN (US); Bruce H. KenKnight, Maple Grove, MN (US)

Correspondence Address: Schwegman, Lundberg, Woessner & Kluth, P.A., P.O. Box 2938, Minneapolis, MN, 55402, US

(21) Appl. No.: 10/722,115

(22) Filed: Nov. 25, 2003

Related U.S. Application Data
(60) Provisional application No. 60/429,954, filed on Nov. 30, 2002.
(60) Provisional application No. 60/483,028, filed on Jun. 27, 2003.

(51) Int. Cl. A61N 1/36

(52) U.S. Cl. 607/3; 435/284.1; 435/285.2

(57) ABSTRACT

A method for cell and electrical therapy of living tissue including administration of exogenous cells into a region of injured tissue and application of electrical energy. In one application the exogenous cells are conditioned in vitro before their administration into tissue. In one application the combined cell and electrical therapy is applied in vivo to damaged heart tissue. In such applications, minimally invasive procedures are used to apply the cell therapy and the electrical therapy is provided via an implantable pulse generator. In one application an implantable pacemaker is used in the VDD or DDD mode with an atrioventricular delay kept relatively short when compared to the intrinsic atrioventricular delay.

### CROSS-REFERENCE TO RELATED APPLICATION(S)

This application claims the benefit of U.S. Provisional Application No. 60/429,954, filed on Nov. 30, 2002 and U.S. Provisional Application No. 60/483,028, filed on Jun. 27, 2003, under 35 U.S.C. § 119(e), which are hereby incorporated by reference.

This application is related to co-pending, commonly assigned U.S. patent application Ser. No. ______, “METHOD AND APPARATUS FOR CELL AND ELECTRICAL THERAPY OF LIVING TISSUE,” filed on Nov. 25, 2003 (Attorney Docket No. 279.466US1), which is hereby incorporated by reference.

### TECHNICAL FIELD

This invention relates generally to combined cell and electrical therapy of living tissue and particularly, but not by way of limitation, to method and apparatus for conditioning living tissue using cell and electrical therapy with a cardiac rhythm management system.

### BACKGROUND

The heart is a unique organ which pumps blood not only to the remaining portions of the body, but to itself. “Heart attacks” or myocardial infarctions occur when there is a loss of proper blood flow to the heart. When heart tissue does not get adequate oxygen, there is a high probability that heart muscle cells will die. The severity of a myocardial infarction is measured by the amount and severity of heart damage and loss of function.

Heart disease is a leading cause of death. Despite advances in the treatment of myocardial infarction, patients suffer decreased quality of life due to the damage caused by the heart attack. One such damage is chronic heart failure arising from the myocardial infarction. The cardiac muscle cells, cardiomyocytes, which, in some circumstances, die during a myocardial infarction either cannot be regenerated naturally by the heart or cannot be regenerated in sufficient quantities to repair the damage following infarction. Depending on the severity of damage to the heart muscle, cardiac output, heart valve function, and blood pressure generating capacity can be greatly reduced. These results only exemplify some of the long-term devastating impacts of heart attacks on patients.

One way to treat damaged heart muscle cells is to provide pharmaceutical therapies in an effort to restore heart function. Such therapies may not be particularly effective if the damage to the heart is too severe, and pharmaceutical therapy is not believed to regenerate cardiomyocytes, but instead acts to block or promote certain molecular pathways that are thought to be associated with the progression of heart disease to heart failure.

Another treatment for damaged heart muscle is called “cell therapy.” Cell therapy involves the administration of endogenous, autologous and/or or nonautologous cells to a patient. For example, myogenic cells can be injected into damaged cardiac tissue with the intent of replacing damaged heart muscle or improving the mechanical properties of the damaged region. However, the administration of myogenic cells does not ensure that the cells will engraft or survive, much less function and there is a need in the art for enhanced efficacy of cell therapies.

### SUMMARY

This document discloses, among other things, a method and apparatus for synergistic actions among cell and electrical therapies of living tissue. In varying embodiments, the present disclosure includes a system for electrical therapy of cardiac tissue of a heart, at least a portion of the cardiac tissue administered with exogenous cells in a cell therapy, the system comprising one or more catheter leads with electrodes and a pulse generator including an interface for connection to the one or more catheter leads, a controller programmable for a plurality of pulse delivery modes, and a sense amplifier for sensing electrical signals from the one or more catheter leads, wherein the pulse generator includes a selectable pacing mode for providing therapeutic electrical stimulation to enhance the cell therapy of the cardiac tissue.

In various embodiments, the therapeutic electrical stimulation includes a VDD (or DDD) pacing mode having an atrioventricular delay which is short compared to an intrinsic atrioventricular delay of the heart.

Also described are embodiments where the therapeutic electrical stimulation is provided at times between additional pacing and defibrillation therapies, where the therapeutic electrical stimulation is programmable for certain times of day, such as during sleep.

Also described are embodiments where the therapeutic electrical stimulation is programmable for certain levels of stress, or for certain levels of activity.

A variety of embodiments are provided where the therapy is invoked by a programmer, where accelerometer data is used to determine when to apply therapeutic electrical stimulation and where lead location is used to determine types of therapeutic electrical stimulation, for some examples.

Also discussed are methods for enhancing cell therapy of cardiac tissue including applying electrical therapy using an implantable pulse generator to cardiac tissue administered with exogenous cell therapy comprising donor cells, wherein the electrical therapy enhances one or more of engraftment, survival, proliferation, differentiation or function of the donor cells. Different methods including in vivo and in vitro treatments are discussed. Various pacing therapies are also discussed. In one embodiment, the methods include administering an agent that enhances exogenous cell engraftment, survival, proliferation, differentiation, or function. Enhancement of cardiac function and angiogenesis are also discussed.

The description also provides various catheters for cell therapy, including needle means for injection of fluids for cell therapy.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

In the drawings, like numerals describe similar components throughout the several views. Like numerals having different letter suffixes represent different instances of the components.

FIG. 1A is a flow diagram showing an overall therapy using cell therapy and electrical therapy according to one embodiment of the present invention.

FIG. 1B is a flow diagram showing an overall therapy using cell therapy and electrical therapy according to one embodiment of the present invention.

FIG. 1C is a flow diagram showing a particular therapy for treating cardiac tissue using combined cell and electrical therapies according to one embodiment of the present invention.

FIG. 1D is a flow diagram showing a particular therapy for treating cardiac tissue using combined cell and electrical therapies according to another embodiment of the present invention.

FIG. 2A is a drawing of a side view of a catheter tip for providing cell therapy according to one embodiment of the present invention.

FIG. 2B is a drawing of a top view of a catheter tip for providing cell therapy according to one embodiment of the present invention.

FIG. 2C is a side view of one embodiment of a catheter tip with adjustable curvature according to one embodiment of the present invention.

FIG. 2D is a drawing of a side view of a catheter tip with separate channels for vacuum and needle for providing cell therapy according to one embodiment of the present invention.

FIG. 2E is a drawing of a side view of a catheter tip with drug reservoir and electrodes for providing cell therapy according to one embodiment of the present invention.

FIG. 2F is a drawing of a catheter tip having a needle array for providing cell therapy according to one embodiment of the present invention.

FIG. 2G is a drawing of the catheter tip of FIG. 2F with the needle array retracted and tissue after cell therapy according to one application of the present invention.

FIG. 2H is a drawing of a catheter tip with expandable balloon for cell therapy according to one embodiment of the present invention.

FIG. 3 is a block diagram of a pacemaker according to one embodiment of the present invention.

FIG. 4 shows one example of application of cell and electrical therapy to a region of cardiac tissue subject to myocardial infarction according to one embodiment of the present invention.

FIG. 5A is a diagram showing a programmer for use with an implanted cardiac rhythm management device according to one embodiment of the present invention.

FIG. 5B is a diagram showing a wireless device in communications with an implanted device for management of the implanted device and therapy according to one embodiment of the present invention.

FIG. 5C is a diagram showing a wireless device in communications with an implanted device and connected to a network for communications with a remote facility for management of the implanted device and therapy according to one embodiment of the present invention.

FIG. 6A is a block diagram showing an in vitro cell treatment device according to one embodiment of the present invention.

FIG. 6B is a block diagram showing additional details of portions of the in vitro cell treatment device according to one embodiment of the present invention.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their equivalents.

It should be noted that references to “an”, “one”, or “various” embodiments in this disclosure are not necessarily to the same embodiment, and such references contemplate more than one embodiment.

Definitions

By “muscle cell” or “muscle tissue” is meant a cell or group of cells derived from muscle, including, but not limited to, cells and tissue derived from skeletal muscle and cardiac muscle, and in some embodiments includes smooth muscle cells. The term includes muscle cells both in vitro and in vivo. Thus, for example, an isolated cardiomyocyte would constitute a “muscle cell” for purposes of the present invention, as would a muscle cell as it exists in muscle tissue present in a subject in vivo. The term also encompasses both differentiated and nondifferentiated muscle cells, such as myocytes, myotubes, myoblasts, both dividing and differentiated, cardiomyocytes and cardiomyoblasts.

By “cardiac cell” is meant a differentiated cardiac cell (e.g., a cardiomyocyte) or a cell committed to differentiating to a cardiac cell (e.g., a cardiomyoblast or a cardiomyogenic cell).

A “myocyte” is a muscle cell that contains myosin.

A “cardiomyocyte” is any cell in the cardiac myocyte lineage that shows at least one phenotypic characteristic of a cardiac muscle cell. Such phenotypic characteristics can include expression of cardiac proteins, such as cardiac sarcomeric or myofilbrillar proteins or atrial natriuretic factor (ANP), or electrophysiological characteristics. Cardiac sarcomeric or myofibrillar proteins include, for example, atrial myosin heavy chain, cardiac-specific ventricular myosin heavy chain, desmin, N-cadherin, sarcomeric actin, cardiac troponin I, myosin heavy chain, and Na/K ATPase. Electrophysiological characteristics of a cardiomyocyte include, for example, Na⁺ or K⁺ channel currents. Similarly, by “skeletal muscle cell” is meant any cell in the skeletal muscle cell lineage that shows at least one phenotypic characteristic of a skeletal muscle cell. Such phenotypic characteristics can include expression of skeletal muscle proteins, such as skeletal muscle-specific transcription factor MyoD or skeletal muscle-specific myosin, or electrophysiological characteristics and morphologic characteristics such as fusion into a multinucleated striated fiber.

By “myocardium” is meant the muscular portion of the heart. The myocardium includes three major types of muscle fibers: atrial muscle fibers, ventricular muscle fibers, and specialized excitatory and conductive muscle fibers.

A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest for gene therapy. Vectors include, for example, viral vectors (such as adenoviruses, adeno-associated viruses (AAV), lentiviruses, herpesvirus and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described (see, e.g., Curiel et al., Proc. Natl. Acad. Sci. USA, 88:8850 (1991)).

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene may include a gene which is partly or entirely heterologous (i.e., foreign) to the transgenic organism, or may represent a gene homologous to an endogenous gene of the organism.

By “transgenic cell” is meant a cell containing a transgene. For example, a stem cell transformed with a vector containing an expression cassette can be used to produce a population of cells having altered phenotypic characteristics.

The term “wild-type” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “transduction” denotes the delivery of a polynucleotide to a recipient cell either in vivo or in vitro, via a viral vector and preferably via a replication-defective viral vector, such as via a recombinant AAV.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “gene,” “polynucleotide,” “coding region,” or “sequence” which “encodes” a particular protein, is a nucleic acid molecule which is transcribed and optionally also translated into a gene product, i.e., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

By “enhancer element” is meant a nucleic acid sequence that, when positioned proximate to a promoter, confers increased transcription activity relative to the transcription activity resulting from the promoter in the absence of the enhancer domain.

By “cardiac-specific enhancer element” is meant an element, which, when operably linked to a promoter, directs gene expression in a cardiac cell and does not direct gene expression in all tissues or all cell types. Cardiac-specific enhancers of the present invention may be naturally occurring or non-naturally occurring. One skilled in the art will recognize that the synthesis of non-naturally occurring enhancers can be performed using standard oligonucleotide synthesis techniques.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide or polypeptide refers to a nucleic acid sequence, peptide or polypeptide that is identified and separated from at least one contaminant nucleic acid, polypeptide or other biological component with which it is ordinarily associated in its natural source. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “peptide”, “polypeptide” and protein” are used interchangeably herein unless otherwise distinguished.

By “growth factor” is meant an agent that, at least, promotes cell growth or induces phenotypic changes.

The term “angiogenic growth factor” means an agent that alone or in combination with other agents induces angiogenesis, and includes, but is not limited to, fibroblast growth factor (FGF), vascular endothelial growth factor (VEGF), hepatocyte growth factor, angiogenin, transforming growth factor (TGF), tissue necrosis factor (TNF, e.g., TNF-α), platelet derived growth factor (PDGF), granulocyte colony stimulatory factor (GCSF), placental GF, IL-8, proliferin, angiopoietin, e.g., angiopoietin-1 and angiopoietin-2, thrombospondin, ephrin-A1, E-selectin, leptin and heparin affinity regulatory peptide.

General Overview

This document describes, among other things, method and apparatus for cell therapy and electrical conditioning of living tissue. In one embodiment, cell therapy is applied to tissue in vivo by locating damaged tissue and administering, e.g., inserting or applying, appropriate cellular material (“donor cells”) into and/or to the damaged tissue. In one embodiment, the area including the damaged tissue and donor cells are then subjected to electric conditioning, such as pacing-level electrical stimulation, using a pulse generator with properly positioned electrodes. Several embodiments are presented below to provide examples of different therapy apparatus and method. It is understood that other apparatus and method are possible as provided by the attached claims and their equivalents.

The invention includes subjecting cells in vitro to one or more stimuli (“in vitro conditioning”) which preferably yields cells with a desirable phenotype for cell-based therapies (“donor cells” hereinafter). For example, cells subjected to in vitro conditioning may be employed in cell-based therapies to augment and/or replace cardiac muscle, for instance, to treat myocardial infarction as well as to treat genetic, e.g., degenerative, and acquired, e.g., heart failure, cardiac disorders. In one embodiment, donor cells compatible with a recipient and subjected to in vitro conditioning, when employed in cell-based therapies to augment and/or replace cardiac muscle in a recipient, result in enhanced cellular engraftment, survival, proliferation, differentiation, cardiac function, and/or angiogenesis as a result of in vitro conditioning and/or in vivo pacing.

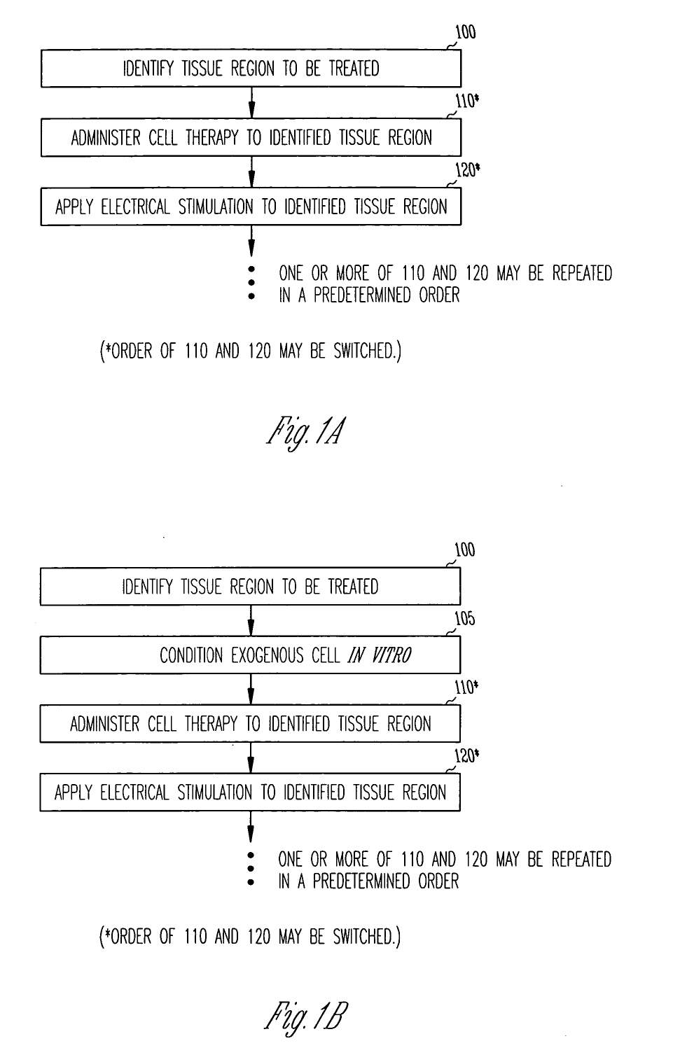

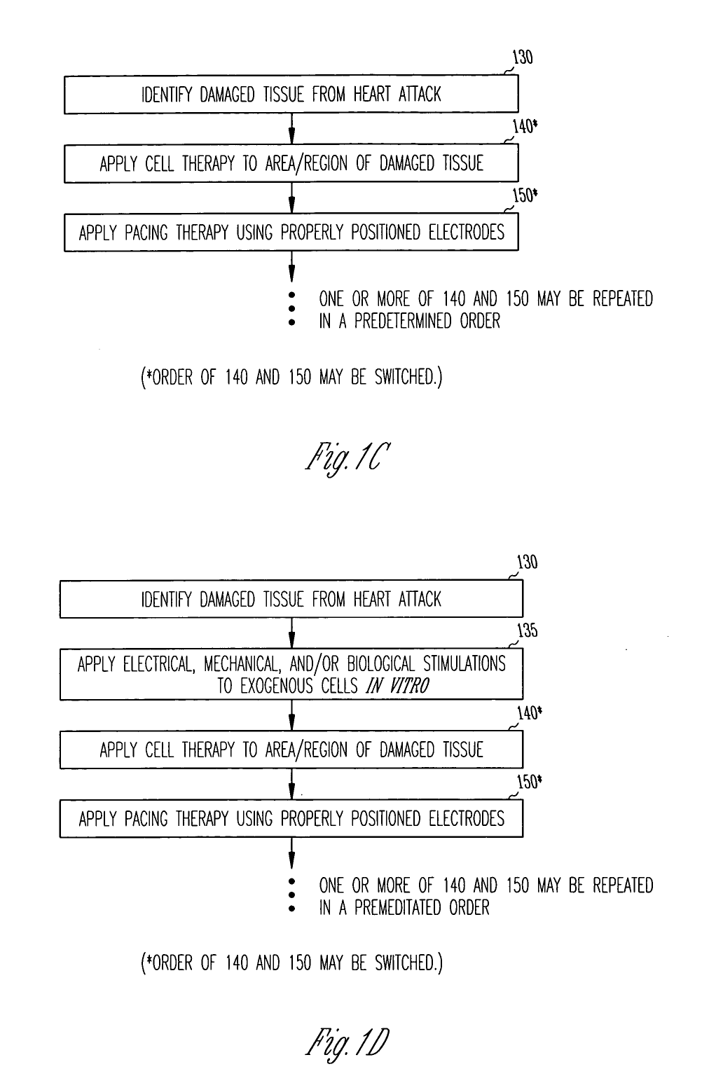

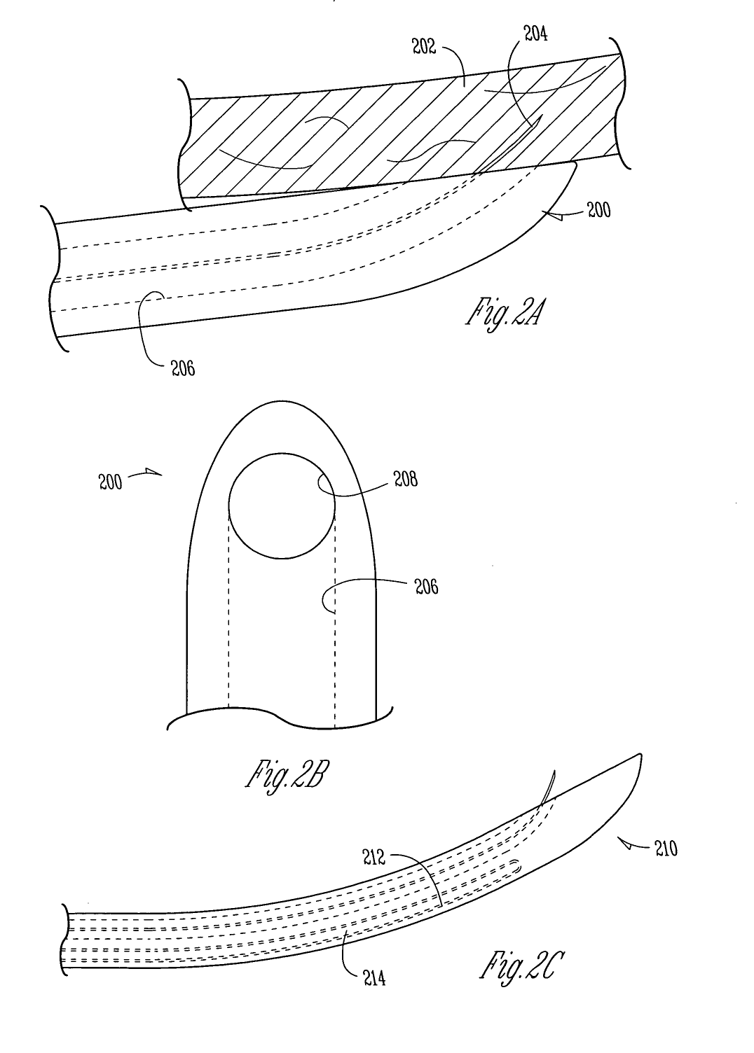

FIG. 1A shows a flow chart for providing combined cell and electrical therapy according to one embodiment of the present invention. A region of the tissue to be treated is identified at 100. Cell therapy is administered to the identified region at 110. Electrical therapy is applied to the identified region at 120. In one approach, the cells (“donor” cells) are administered concurrently with electrical therapy, while in other approaches electrical therapy is subsequent to cell administration. In another approach electrical therapy is applied prior to cell administration. Moreover, it is understood that multiple cell therapies may be implemented prior to application of the electrical therapy to the identified tissue region. Also for example, the cell therapy may be followed by multiple electrical therapies. It is understood that different permutations of cell and electrical therapy may be performed in varying embodiments. For instance, electrical conditioning may be applied before, during, or after cell therapy. In one approach cellular engraftment, cellular proliferation, cellular differentiation, cellular survival and/or cellular function, e.g., contractile function, of the donor cells in the recipient is further enhanced by electrical stimulus from the electrical therapy.

FIG. 1B shows a flow chart for providing combined cell and electrical therapy according to another embodiment of the present invention. This embodiment includes an additional step of preparing the donor cells before administering the cell therapy. The donor cells are conditioned in vitro to introduce one or more desirable gene products (transgenes) to the cells at 105. Preferably, the transgenic donor cells include a transgene that enhances cellular proliferation, cellular engraftment, cellular survival, cellular differentiation and/or cellular function of the donor cells in the recipient. The expression of one or more transgenes may be employed to decrease, replace or supplement (increase) the expression of endogenous genes in the donor cells, e.g., if the donor cells are autologous cells and the donor has an inherited or acquired disease associated with aberrant expression of an endogenous gene in cardiac cells. The expression of one or more transgenes may correct the level of the gene product encoded by the transgene in the donor cells. In one embodiment the expression of the transgene is controlled by a regulatable or tissue-specific, e.g., cardiac myocyte-specific promoter. The transgene may be introduced to donor cells by any means including but not limited to liposomes, electroporation, naked DNA, or viral-mediated transduction, for instance, via adenovirus, adeno-associated virus, retrovirus or lentivirus vectors.

In one embodiment an advanced patient management device is used to control the applied electrical therapy in conjunction with inputs regarding applied cell therapy, inputs regarding patient health, and inputs regarding environmental conditions. Other inputs are contemplated, and those provided herein are intended to demonstrate the flexibility and programmability afforded the user when the cell and electrical therapies are managed with an advanced patient management system. Such a system is discussed in various applications by the assignee, including, but not limited to, in U.S. patent application Ser. No. 10/093,353, filed Mar. 6, 2002, which is hereby incorporated by reference in its entirety.

### EXAMPLE OF CELL THERAPY OF CARDIAC TISSUE

The present teachings are useful in a number of therapies. In one example, the treatment of a failing heart is possible. Such therapies may be employed for both ischemic and non-ischemic heart failure etiologies.

FIG. 1C is a flow diagram showing a particular therapy for treating cardiac tissue using combined cell and electrical therapies according to one embodiment of the present invention. The cardiac tissue region (or regions) of damaged tissue are identified at 130 and then cell therapy is applied to one or more areas of damaged tissue at 140. Pacing therapy is applied to the identified cardiac tissue region at 150. Tissue damage resulting from a myocardial infarction or heart attack is one type of tissue treatable by these apparatus and methods.

Different methods of locating the damaged tissue may be employed. For example, electrophysiology, such as electrocardiograms, can be used to locate damaged cardiac tissue. Other locating methods include, but are not limited to: echocardiography and catheter-based voltage mapping of a portion of the heart; catheter based strain mapping; invasive or minimally invasive surgery (visualization of damaged tissue); and other imaging techniques, such as MRI, perfusion imaging, fluoroscopy, and angiography.

Once the damaged tissue is located, the localized area may be treated by inserting or applying donor cells, e.g., cells administered intravenously, transvenously, intramyocardially or by any other convenient route, and delivered by a needle, catheter, e.g., a catheter which includes an injection needle or infusion port, or other suitable device. Some exemplary delivery apparatus and methods include, but are not limited to, the teachings provided in the patent applications entitled: Drug Delivery Catheter with Retractable Needle, U.S. patent application Ser. No. 09/746,498, filed Dec. 21, 2000; and Intra-Ventricular Substance Delivery Catheter System, U.S. patent application Ser. No. 10/038,788, filed Dec. 31, 2001. Both of these disclosures are incorporated by reference in their entirety.

FIG. 1D is a flow diagram showing a particular therapy for treating cardiac tissue using combined cell and electrical therapies according to another embodiment of the present invention. This embodiment includes an additional step of preparing the donor cells before applying the cell therapy. The donor cells are prepared by electrical, mechanical, and/or biological conditioning in vitro at 135.

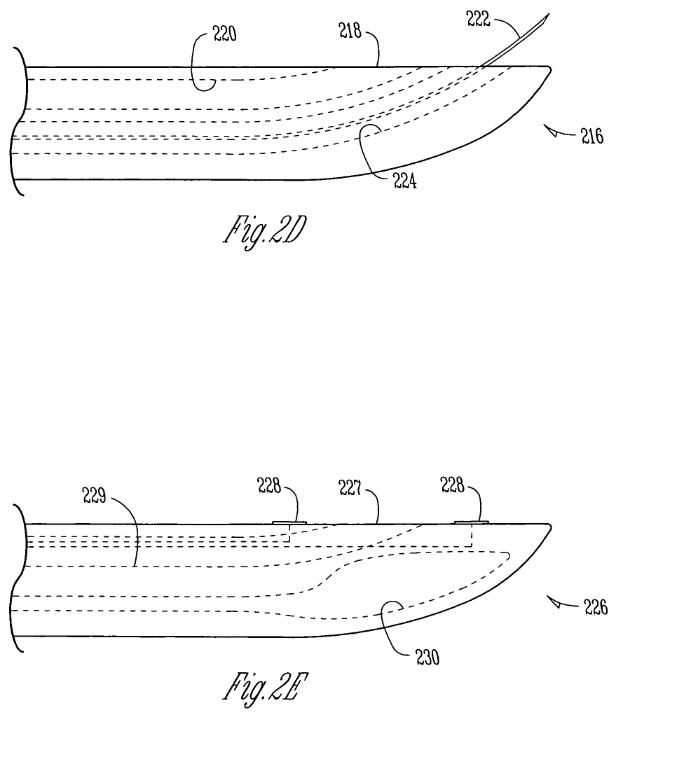

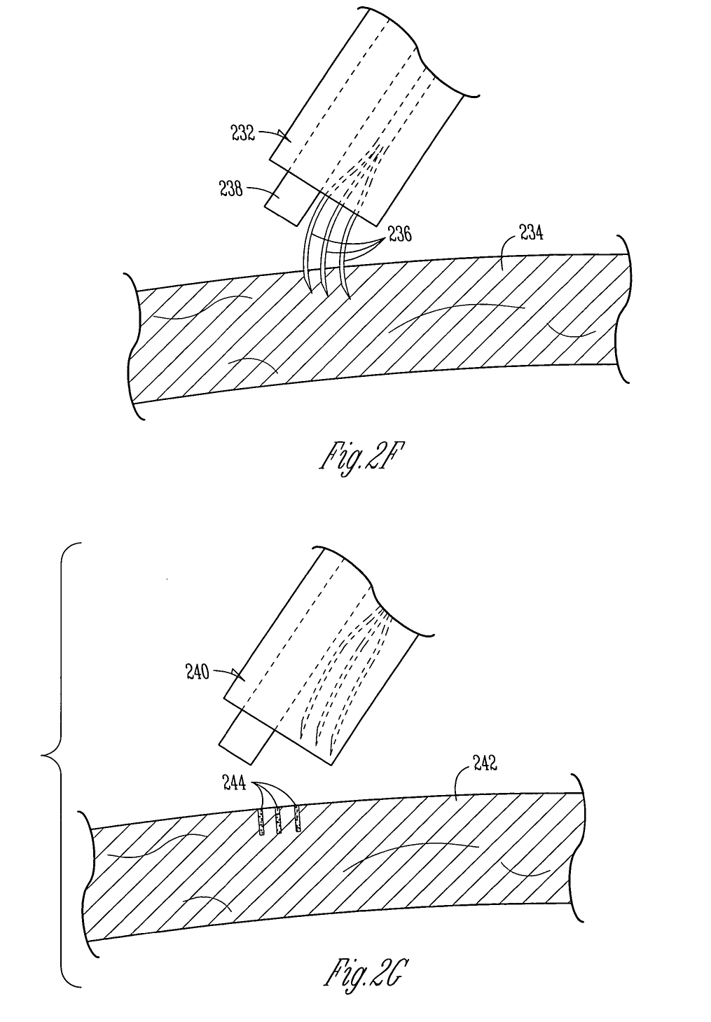

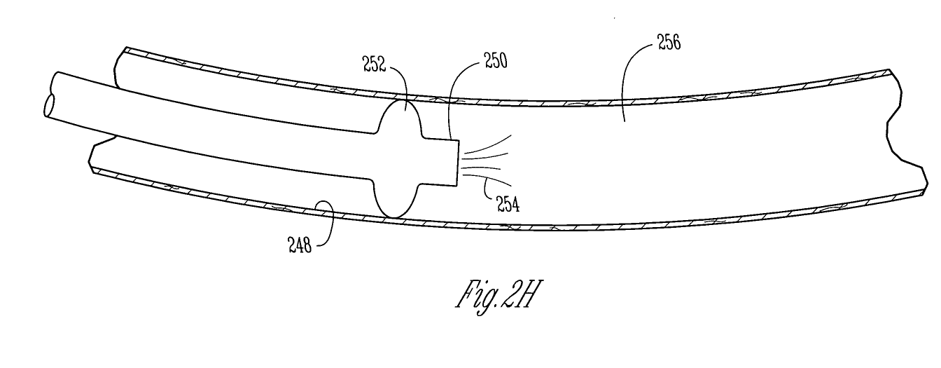

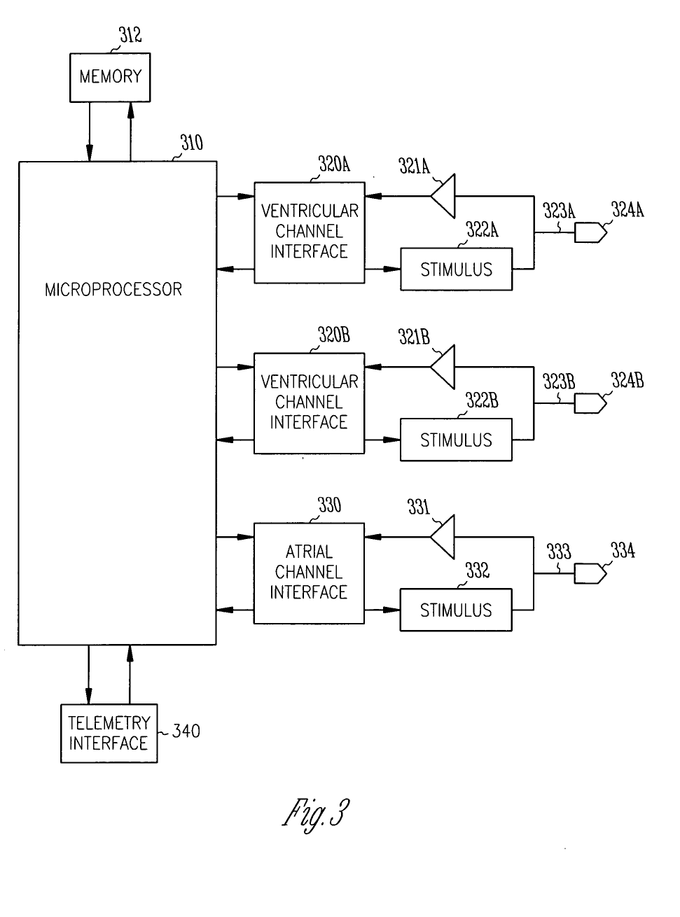

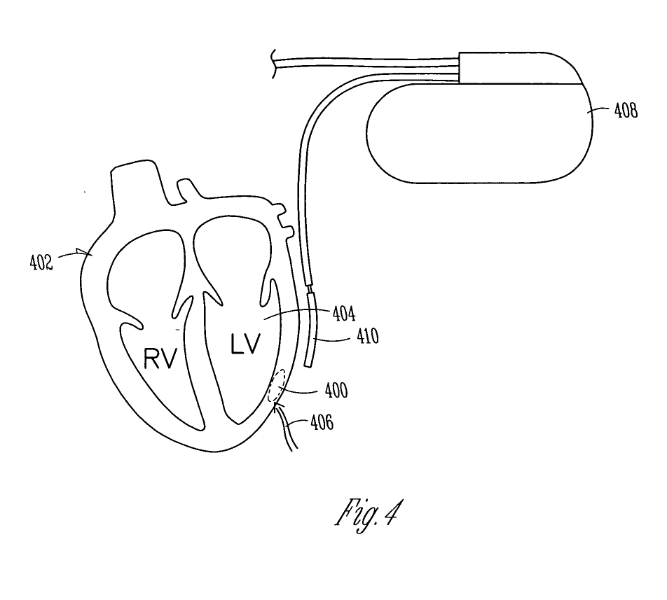

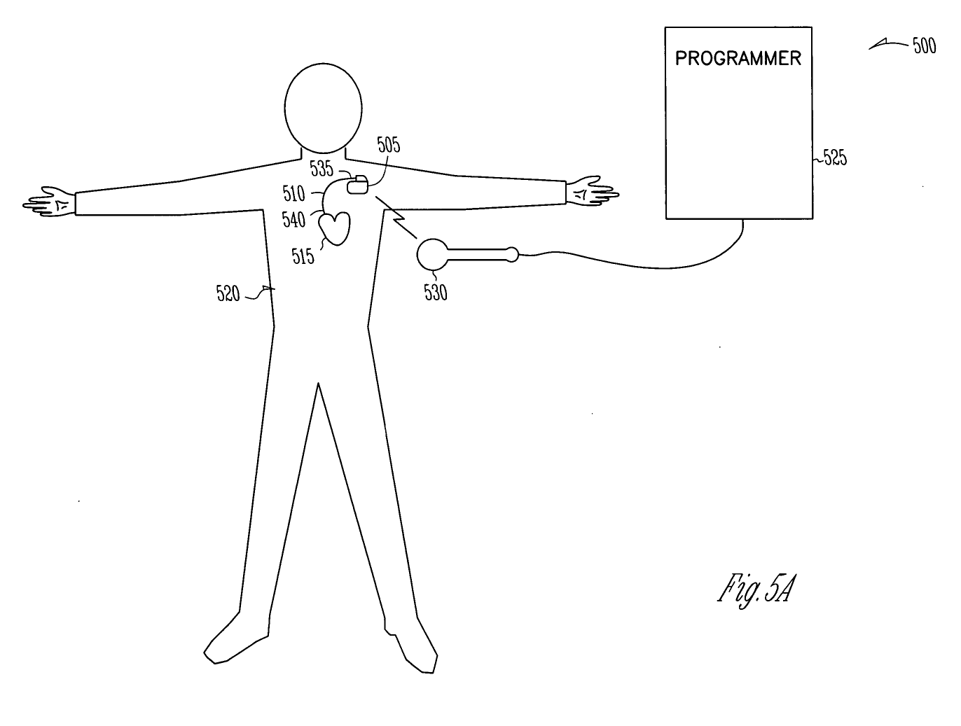

In one embodiment, a catheter having a catheter tip adapted for injection of exogenous cellular material is used for cell therapy. FIG. 2A shows a side view of a catheter tip 200 positioned near the myocardium 202 having damaged cardiac muscle tissue. The catheter tip 200 is positioned intrapericardially intravenously, transvenously, transarterially, intramyocardially, or by another method. A suction port 208 is shown from a top view in FIG. 2B at the distal end of the catheter. The catheter tip 200 is affixed near the region to be treated by a vacuum applied at the proximal end of the catheter to create a vacuum at the suction port 208 via channel 206 and thereby hold the catheter tip 200 against the myocardium 202. A hollow needle 204 is then advanced into the tissue at the catheter tip to inject exogenous cellular material to the location for cell therapy. After injection is complete, the hollow needle 204 is retracted into catheter tip 200 and the vacuum is removed so that the catheter tip 200 can be repositioned for therapy at a different location.

In one embodiment, the needle is deployed through a channel and using an actuator at the proximal end of the catheter. In the example where a common channel is used between the vacuum and the needle, the vacuum channel is sealed where the needle exits the catheter at the proximal end to maintain any vacuum applied to the channel. The hollow needle in this embodiment uses a conduit from the proximal end to the distal end of the catheter. In one embodiment, injection of fluid is accomplished using a luer fitting and needle at the proximal end. Manipulation of the needle is accomplished using the actuator at the proximal end of the catheter.

The example demonstrated in FIG. 2A employs channel 206 for both the application of vacuum and a means for guiding hollow needle 204 and storing it when it is retracted. Other embodiments are provided herein where the suction port and needle use separate channels. For example, FIG. 2D shows a catheter tip 216 having suction port 218 with channel 220 and a hollow needle 222 with channel 224. In this example embodiment, channel 220 and channel 224 are separate channels. Other configurations are possible without departing from the scope of the present teachings.

In the embodiment with separate channels, a separate fitting for the vacuum and for the needle are used to apply the vacuum and inject fluid, respectively. In one embodiment a standard luer fitting is used and the needle is used to inject the fluid.

FIG. 2C shows one example of an embodiment where the catheter tip 210 is able to achieve an angle of curvature to provide a surface that conforms to a portion of a curved myocardium. In one embodiment, the angle of curvature is approximately 30 degrees. In varying embodiments the tip may be adjusted to perform differing degrees of deflection to adjustably position the suction port near the location to be treated. In one embodiment, the adjustment is performed using a stylet inserted into a pre-bent catheter tip portion. FIG. 2C demonstrates this by including a stylet channel 212 which accommodates a stylet 214 in varying positions to show that as the stylet is removed, the angle of the tip changes and is thus adjustable. Other adjustment techniques may be employed without departing from the scope of the present teachings.

FIG. 2E shows one embodiment of a catheter having a catheter tip 226 at the distal end. The catheter tip 226 includes one or more contact electrodes 228 connected to the proximal end and a drug reservoir 230 with elution means to perform iontophoresis. Various locations of possible electrode positions are demonstrated in FIG. 2E. The catheter includes a vacuum channel 229 that terminates in an suction port 227 at the catheter tip 226. The catheter tip 226 is affixed near the region to which drug is delivered by a vacuum applied at the proximal end of the catheter to create a vacuum at the suction port 227 via channel 229. In one embodiment, a chemical reservoir is included at the catheter tip 226 for iontophoretic transfer into the adjacent tissue. In one embodiment, a porous electrode is used to transfer fluid from the catheter tip.

In varying embodiments, the catheter is dimensioned for different sizes to facilitate transvenous positioning of the cathode tip. In one embodiment, the catheter is available in diameters varying from 10 French to 24 French. Other sizes are possible without departing from the present teachings.

Another embodiment of a catheter tip for injection of exogenous cells is shown in FIG. 2F. In this example, the catheter tip 232 includes a needle array 236 which provides a plurality of needle points for injection into tissue 234. The needle array provides multiple pathways for delivery of material and lower delivery resistance. The catheter tip 232 also includes fiber optic 238 for visualizing the region and locating the catheter tip 232 for treating tissue 234.

In one embodiment, the needle array 236 is retractable for ease of transvenous and transarterial delivery. In one embodiment, the needle array 236 includes needle points that are approximately 0.5 cm in length. In varying embodiments, the needle array includes needles of varying lengths to provide a contour of tip points. In varying embodiments the needle array provides 2-3 mm of penetration into tissue. Other embodiments are possible without departing from the scope of the present teachings.

In one application demonstrated by FIG. 2G, a plurality of columns 244 of material are injected into tissue 242 by catheter tip 240. (The catheter tip 240 is shown in a retracted mode in FIG. 2G.) The columns 244 may contain cellular material and/or drugs and serve as passive molecule factories in tissue 242.

It is understood that the number and placement of tines may vary. Diameters and distances provided herein are intended to provide nonexclusive examples and are not intended in an exclusive or limiting sense.

Another embodiment of a catheter-based delivery system includes the use of a balloon and delivery catheter. FIG. 2H shows one example of a catheter tip 250 which is insertable transvenously and transarterially for the delivery of cellular materials to a vessel or organ. Catheter tip 250 includes balloon 252 for occluding the lumen 248 and providing a temporary blockage for the material 254 to remain in space 256 for a period of time. Space 256 is treated with the cellular material, and then balloon 252 is deflated for withdrawal of the catheter tip 250.

It is understood that various combinations of the examples provided above are possible. For example, a fiber optic may be used to place the catheter tip and may be combined with the catheter tips having common and independent channels for the vacuum and the needle and or needle array. Other combinations are possible without departing from the scope of the present teachings.

Combined cell and electrical therapy may also be accompanied by the administration of drugs.

Variations in design and placement of elements may be implemented without departing from the teachings provided herein, and the examples given are not intended in a limited or exclusive sense.

Sources of Cells for in vitro Conditioning and Subsequent Cell Therapy

Sources for donor cells in cell-based therapies include skeletal muscle derived cells, for instance, skeletal muscle cells and skeletal myoblasts; cardiac derived cells, myocytes, e.g., ventricular myocytes, atrial myocytes, SA nodal myocytes, AV nodal myocytes, and Purkinje cells; bone marrow-derived cells, e.g., mesenchymal cells and stromal cells; smooth muscle cells; fibroblasts; or pluripotent cells or totipotent cells, e.g., teratoma cells, hematopoietic stem cells, for instance, cells from cord blood and isolated CD34⁺ cells, multipotent adult progenitor cells, adult stem cells and embyronic stem cells. In one embodiment, the donor cells are autologous cells including xenologous cells. In another embodiment, the donor cells include non-autologous cells. The donor cells can be expanded in vitro to provide an expanded population of donor cells for administration to a recipient. In addition, donor cells may be treated in vitro to induce certain phenotypic characteristics, e.g., to induce proliferation or differentiation, to introduce one or more transgenes, or a combination thereof. Sources of donor cells and methods of culturing those cells are known to the art. See, for example, U.S. Pat. No. 5,130,141 and Jain et al. (Circulation, 103, 1920 (2001)), wherein the isolation and expansion of myoblasts from skeletal leg muscle is discussed (see also Suzuki et al., Circulation, 104, I-207 (2001), Douz et al., Circulation, III-210 (2000) and Zimmerman et al., Circulation Res., 90, 223 (2002)). Published U.S. application 20020110910 discusses the isolation of and media for long term survival of cardiomyocytes. U.S. Pat. No. 5,580,779 discusses isolating myocardial cells from human atria and ventricles and inducing the proliferation of those myocardial cells. U.S. Pat. No. 5,103,821 discusses isolating and culturing SA node cells. For SA node cells, the cells may be co-cultured with stem cells or other undifferentiated cells. U.S. Pat. No. 5,543,318 discusses isolating and culturing human atrial myocytes. U.S. Pat. Nos. 6,090,622 and 6,245,566 discusses preparation of embryonic stem cells, while U.S. Patent No. 5,486,359 discusses preparation of mesenchymal cells.

Exemplary Methods of Isolating Donor Cells

A. Donor Myoblasts and Myocytes

i. Cardiac Tissue

Cardiomyocytes may be prepared by a modification of established methods. In particular, primary myocardial cell isolation is done by modifying established protocols by Nag and Chen, Tissue Cell, 13, 515 (1981) and Dlugaz et al., J. Cell Biol., 99, 2268 (1984). Briefly, a heart, e.g., from an organ donor, is dissected and washed in media. Digestion media includes modified Jolicks MEM containing 10 mM HEPES, 10 mM pyruvate, 5 mM L-glutamine, 1 mM nicotinamide, 0.4 mM L-ascorbate, 1 mM adenosine, 1 mm D-ribose, 1 mM MgCl₂, 1 mM taurine, 2 mM DL-carnitine, and 2 mM KHCO₃. The hearts are minced in digestion media with 0.5 mg/ml collagenase (Worthington) and 100 mM CaCl₂. The tissue is treated with successive digestions for 15 minutes at 37° C. The cells from the first digestion are discarded and the next six digestion reactions are pooled. Cells are preplated for 1 hour to remove fibroblasts, then plated in PC-1 (Ventrex)/DME-Hams F12 media.

Alternatively, heart muscle is dissected from the left ventricular free wall and quickly cut into pieces of approximately 1 mm³ using an array of razor blades. The pieces are incubated for 12 minutes, while shaking at 37° C. in 25 ml of a solution containing 1-2 μM calcium (LC) 120 mM NaCl, 5.4 mM KCl, 5 mM, MgSO₄, 5 mM pyruvate, 20 mM glucose, 20 mM taurine, 10 mM HEPES, and 5 mM nitrilotriacetic acid, pH 6.96. The medium is changed several (about 3) times during the twelve minutes. The pieces are stirred by bubbling with 100% O₂. After removal of the LC medium by straining with 300 μm gauze, the pieces are incubated at 37° C. for 45 minutes in LC without nitrilotriacetic acid, and 4 U/ml of type XXIV protease and 30 μM calcium added, followed by two 45 minute periods with the protease omitted and 400 IU/ml collagenase added. The medium is shaken under an atmosphere of 100% O₂. At the end of the second and third 45 minute periods, the solution containing the dispersed cells is filtered through a 300 μm gauze and centrifugated at 40×g for 1-2 minutes.

Alternatively, primary ventricular myocytes and cardiac fibroblasts are prepared using a Percoll gradient method as described by Iwaki et al., J. Biol Chem., 265, 13809 (1990). Cardiac fibroblasts are isolated from the upper band of the Percoll gradient, and subsequently plated in high glucose Dulbecco's modified Eagle's medium supplemented with 10% fetal bovine serum. Myocytes are isolated from the lower band of the Percoll gradient and subsequently plated in 4:1 Dulbecco's modified Eagle's medium; 199 medium, 10% horse serum and 5% fetal bovine serum.

After isolation, the cells may be washed in a medium containing calcium, e.g., 30 μM calcium, and resuspended in culturing media. Such culture media can comprise DMEM, BSA, ascorbic acid, taurine, carnitine, creatinine, insulin, penicillin G sodium, and an antibiotic, e.g., DMEM with the addition of 0.2 g BSA, 0.1 mM ascorbic acid, 50 mM taurine, 16 mM carnitine, 50 mM creatine, 0.1 μM insulin, 50 U/ml penicillin G sodium, and 50 mg/ml streptomycin sulfate. Culture media can also comprise DMEM without calcium chloride anhydrous and D-calcium pantothenate.

Omega 3 fatty acids have been shown by Kang & Leaf (Circulation, 94, 1774 (1996)) to protect against calcium overload and calcium paradox. Therefore, the culture media may also comprise omega 3 fatty acids, such as, docosaheanoic acid, eicosapentaenoic acid, eicosatetraynoic acid, or polyunsaturated fatty acid.

Magnesium (Mg⁺) is also known to be protective against calcium overload and has been shown to be beneficial in failing human myocardium (Schwinger et al., Am. Heart J., 126, 1018 (1993); Schwinger et al., J. Pharmacol. Exp. Ther., 263, 1352 (1992)). Therefore, the culture media may comprise varying concentrations of Mg²⁺, e.g., from 0.1 to 16 mM.

In one embodiment, cardiomyocytes are obtained from a tissue sample from a subject, e.g., a vertebrate subject, and successively exposed to a first solution with decreasing amounts of CaCl₂. The first solution further includes NaCl, HEPES, MgCl₂, KCl, and sugar at a pH of approximately 7.4, e.g., 140 mM NaCl, 10 mM HEPES, 1 mM MgCl₂, 5.4 mM KCl, and 10 mM sugar at a pH of approximately 7.4. The tissue may be disassociated with an enzyme solution and repeatedly resuspended in a second solution with increasing amounts of CaCl₂. The second solution may further include Earle's modified salt, L-glutamine, sodium bicarbonate, sodium pentothenate, creatine, taurine, ascorbic acid, HEPES, fetal bovine serum, an antibiotic, and a fatty acid, at a pH of approximately 7.4, e.g., sodium bicarbonate at 1250 mg/l, creatine at 328 mg/500 ml, taurine at 312 mg/500 ml, ascorbic acid at 8.8 mg, HEPES at 2.383 g/500 ml, fetal bovine serum at 10% v/v, an antibiotic at 5% v/v, and a fatty acid at 1 μM at a pH of approximately 7.4.

In yet another embodiment, the second solution can be used to cultivate isolated cells, e.g., cardiomyocytes, including the steps of resuspending the isolated cells approximately every 24 hours in the second solution. In still another embodiment, the second solution can be used as maintenance or culture media for cells, e.g., cardiomyocytes.

In another embodiment, cardiomyocytes are obtained from a tissue sample from a subject, e.g., a vertebrate subject, by cutting the tissue into smaller pieces and incubating the tissue in a first solution. The first solution includes calcium, salts, magnesium sulfate, pyruvate, glucose, taurine, HEPES, and nitrilotriacetic acid, e.g., 1-2 μM CaCl₂, 120 mM NaCl, 5.4 mM KCl, 5 mM MgSO₄, 5 mM pyruvate, 20 mM glucose, 20 mM taurine, 10 mM HEPES, and 5 mM nitrilotriacetic acid, at a pH of approximately 6.96. After the addition of an enzyme, e.g., collagenase, to the first solution, the tissue is further incubated in the solution and later subjected to centrifugation to obtain isolated cells. After shaking the tissue at 37° C. for 12 minutes, and bubbling 100% O₂ through the solution, the tissue is incubated in a second solution comprising 1-2 μM CaCl₂, 30 μM NaCl, 5.4 mM KCl, 5 mM MgSO₄, 5 mM pyruvate, 20 mM glucose, 20 mM taurine, 10 mM HEPES, and 4 U/ml of a digestive enzyme, and subsequently incubated in a third solution comprising approximately 1-2 μM, 30 μM NaCl, 5.4 mM KCl, 5 mM MgSO₄, 5 mM pyruvate, 20 mM glucose, 20 mM taurine, 10 mM HEPES, and 4 U/ml of a digestive enzyme Preferably, 400 U/ml of a digestive enzyme, e.g., a type XXIV protease, such as matrix metalloproteinase 2 or 4, and a collagenase, for example, matrix metalloproteinase 1, 3, or 9, is added to the third solution and the tissue subjected to centrifugation to obtain isolated cells.

Other solutions to enhance the yield and long-term survival rate of isolated cardiomyocytes include those in published U.S. application 20020110910.

ii. Neonatal Skeletal Tissue

To harvest cells from neonatal tissue, muscle tissue is harvested from a limb and placed in a culture dish (65 mm diameter) with 8 ml of calcium-free PBS. Muscles are removed under sterile conditions. All harvested tissue is transferred to a 50 ml conical tube containing 12 ml of tissue dissociation solution (TDS) (DMEM with 5% by weight dispase and 0.5% by weight collagenase IV) and stirred for approximately one hour in order to dissociate the tissue. The tube is then centrifuged at 1200×g for approximately 15 minutes. After removal of the supernatant, cells are resuspended in 20 ml of Ham's F12 with 20 mg of collagenase type IV and incubated at 37° C. for one hour to allow tissue dissociation. The tube is again centrifuged at 1200 g for 15 minutes, after which the supernatant is removed and the cells are resuspended in growth media (GM) (400 ml F12, 100 ml FBS and 100 U/ml penicillin G). Within this cell suspension will likely be fibroblasts in addition to myogenic precursor cells.

iii. Adult or Aged Skeletal Tissue

Skeletal muscle may also be harvested from adult tissue and cut into strips. Unlike neonatal tissue, muscle tissue from adult or aged animals yields more satellite cells if initially preincubated before complete tissue dissociation. The increased activation of satellite cells may result from the use of NaN₃ in the preincubation media (PI) (90 ml DM and 10 ml 0.05% NaN₃ in 0.9% saline, where DM is 465 ml DMEM, 35 ml horse serum and 100 U/ml penicillin G).

To preincubate the muscle tissue, the strips are pinned in a SYLGARD™ coated culture dish (35 mm diameter), covered with 2.5 ml of PI, and sterilized by exposure to ultraviolet light for approximately 40 minutes. The dishes are then maintained at 37° C. in a water-saturated atmosphere containing 5% CO₂ for 24 to 72 hours, where optimal pre-incubation times may vary for different muscles.

After pre-incubation, each muscle strip is placed into a 50 ml conical tube with 15 ml TDS solution and incubated in a shaker bath at 37° C. for approximately 3 hours until complete dissociation is observed. Immediately upon complete tissue dissociation, the tubes are centrifuged at 1200 g for 15 minutes. Subsequently, the supernatant is aspirated and cells are reconstituted with 5 ml GM. As with the cells derived from neonatal tissue, fibroblasts may be included in the cell suspension.

Alternatively, myogenic cells are released from skeletal muscle fragments by serial enzyme treatments. A one hour digestion with 600 U/ml collagenase (Sigma, St. Louis, Mo., USA), is followed by a 30 minute incubation in Hank's balanced salt solution (HBSS) containing 0.1% w/v trypsin (Gibco Lab, Grand Island, N.Y., USA). Satellite cells are placed in 75 cm² culture flasks (Coster, Cambridge, Mass., USA) in proliferation medium, e.g., 199 medium (Gibco Lab.) with 15% fetal bovine serum (Gibco), 1% penicillin (10,000 U/ml) and 1% streptomycin (10,000 U/ml).

In particular, for human myoblasts, these cells are grown from donor human muscle and passaged cells are seeded at 2-3,000 cells per well in a 96 well cluster plate in Ham F12 medium containing 7.5% up to 20% v/v FCS. The medium may contain varying concentrations of LIF. Cell numbers are counted at times up to 12 days. There is a marked stimulation of proliferation of myoblasts by LIF, e.g., at 30 U/ml. FGF and HBGF also stimulate growth of satellite cells (DiMario et al., Differentiation, 39, 42 (1988)). TGF-α also stimulates human cells at concentrations ranging up to 10 ng/ml.

In one embodiment, to expand skeletal muscle cells, skeletal muscle cells are cultured with isolated PDGF, TGF-beta, and/or FGF, e.g., at 5-10 ng/ml.

B. Non-Muscle Donor Cells

Methods to isolate and/or culture non-muscle donor cells, and methods to induce a muscle cell-specific phenotype to those cells, i.e., differentiation, are known to the art. For instance, mesenchymal stem cells may be obtained by culturing adherent marrow or periosteal cells. To induce a cardiac cell-specific phenotype, MSCs cells may be cocultured with fetal, neonatal or adult cardiac cells optionally in the presence of fusigens, extracts of mammalian hearts, one or more growth factors, one or more differentiating agents, or subjected to mechanical or electrical stimulation.

Bone marrow is a source for donor cells which have the potential to differentiate into cardiomyocytes. To obtain bone marrow cells, a bone marrow puncture is conducted by sternal or iliac puncture. After skin disinfection of the part for puncture, a donor is subjected to local anesthesia. Particularly, subpeiosteum is thoroughly anesthetized. The inner tube of a bone marrow puncture needle is pulled out and a 10 ml syringe containing 5000 U of heparin is attached to the needle. Normally 10-20 ml of the bone marrow fluid is quickly taken by suction and the puncture needle is removed, followed by pressure hemostasis for about 10 minutes. The obtained bone marrow fluid is centrifuged at 1000×g to recover bone marrow cells, which are then washed with PBS (phosphate buffered saline). After this centrifugation step is repeated twice, the obtained bone marrow cells are suspended in a cell culture medium such as A-MEM (a-modification of MEM), DMEM (Dulbecco's modified MEM) or IMDM (Isocove's modified Dulbeccos's medium) each containing 10% FBS (fetal bovine serum) to prepare a bone marrow cell suspension.

For the isolation of the bone marrow cells having the potential to differentiate into cardiomyocytes from the obtained bone marrow cell suspension, any method can be employed, so long as it is effective at removing other cells existing in the cell suspension such as hematocytes, hematopoietic stem cells, vascular stem cells and fibroblasts. For example, based on the method described in Pittenger et al., Science, 284, 143 (1999), the desired cells can be isolated by subjecting the cell suspension layered over Percoll having the density of 1.073 g/ml to centrifugation at 1100×g for 30 minutes, and the cells on the interface are recovered. Furthermore, a bone marrow cell mixture containing the cells having the potential to differentiate into cardiomyocytes can be obtained by mixing the above cell suspension with an equal amount of Percoll solution diluted to 9/10 with 10×PBS, followed by centrifugation at 20000×g for 30 minutes, and recovering the fraction having the density of 1.075-1.060. A bone marrow cell mixture is diluted into single cell using 96-well culture plates to prepare a number of clones respectively derived from single cells. The clones having the potential to differentiate into cardiomyocyte can be selected by the observation of spontaneously beating cells generated by the treatment.

Umbilical blood is another source for donor cells. To prepare those cells, umbilical blood is separated from the cord, followed by addition of heparin to give a final concentration of 500 U/ml. After thoroughly mixing, cells are separated from the umbilical blood by centrifugation and resuspended in a cell culture medium, such as α-MEM, DMEM or IMDM, each containing 10% FBS. From the cell suspension thus obtained, cells having the potential to differentiate into cardiomyocytes can be separated using, for example, antibodies.

Fibroblasts are also a source for donor cells of the invention.

In vitro Conditioning

A variety of exogenous stimuli (“conditioning”) may be employed in the methods of the invention. For instance, donor cells may be treated in vitro by subjecting them to mechanical, electrical, or biological conditioning, or any combination thereof. The conditioning may include continuous or intermittent exposure to the exogenous stimuli. Preferred exogenous agents include those which enhance the survival, engraftment, differentiation, proliferation and/or function of donor cells after transplant. One example of a cell treatment device for such in vitro conditioning is described later in this document.

A. Mechanical Conditioning

Mechanical conditioning includes subjecting donor cells to a mechanical stress that simulates the mechanical forces applied upon cardiac muscle cells in the myocardium due to the cyclical changes in heart volume and blood pressure. In one embodiment, a cyclic mechanical stress is applied to the donor cells. In one embodiment, the cyclical mechanical stress applied to donor cells results in the cyclical deformation of these cells, resembling the cyclical deformation (contraction) of cardiac muscle cells in vivo. The mechanical stress includes subjecting one or more donor cells, preferably a population of donor cells, to a mechanical force in one dimension and in one direction, or alternatively, in one dimension and in two or more opposite directions, for example, causing the donor cells to stretch and relax at a predetermined frequency for a predetermined duration. Mechanical conditioning can result in donor cells that are capable of contracting upon excitation by action potentials.

Mechanical conditioning preferably alters gene expression, protein synthesis, and/or the activity of one or more cellular kinases in donor cells, and in one embodiment results in proliferation and/or differentiation of the donor cells. In one embodiment, mechanical conditioning of donor cells results in an altered expression profile, e.g., an altered expression profile for genes encoding BMP, VEGF, angiotensin II, and the like, in the donor cells. In one embodiment, mechanical conditioning of donor cells results in an increase in the number and/or activity of contractile elements including actin and myosin filaments, which are protein structures that interact with each other during muscle contraction. Donor cells subjected to mechanical conditioning thus develop contractility that is characteristic of muscle cells.

In one embodiment, the mechanical conditioning includes subjecting donor cells to a mechanical force so that the donor cells are physically extended in at least one direction by approximately 5% to 20% of their length, and at a frequency of 0.25 to 2 Hz. In other words, at least one donor cell is forced to increase its length by 5% to 20% at 0.25 to 2 times per second. This simulates the mechanical tension which cardiac muscle cells are subject to under physiological conditions in vivo. In one embodiment, donor cells are plated on a controllably deformable culturing substrate in the presence of culturing media. The substrate is cyclically deformed to simulate the mechanical displacement of cardiac muscle. In one specific embodiment, the substrate includes a distensible strip made of medical grade silicone. Donor cells are plated on the distensible strip. The distensible strip is stretched and released, such that the donor cells on it change their length with the distensible strip in a manner simulating the cardiac muscle cells in vivo. One example of such an apparatus for applying mechanical stress to cells in a culture is given in Terracio et al., In Vitro Cellular & Developmental Biology, 24(1), 53-58, 1988, where the silicone strip is subject to calibrated mechanical tension created with a variable speed motor.

In one embodiment, the mechanical conditioning is applied continuously for a predetermined period of time. In one specific embodiment, the predetermined period is in the range of 1 to 14 days. In another embodiment, the mechanical conditioning is applied intermittently for a predetermined period of time interrupted by one or more resting (non-stimulating) periods. In one specific embodiment, the mechanical conditioning is applied with a duty cycle that is in the range of 5% to 75% for a predetermined period that is in the range of 1 to 14 days.

B. Electrical Conditioning

Electrical conditioning includes subjecting donor cells to electrical conditions that simulate the electrical conditions in the myocardium which result in contraction of the heart. In the heart, contraction results primarily from the contractions of atrial and ventricular muscle fibers. Contraction of atrial and ventricular muscle fibers is slower and is of a longer duration than the contraction of skeletal muscle. Cardiac muscle and skeletal muscle, however, share a number of common anatomic characteristics. In the same manner as skeletal muscle, cardiac muscle is made up of elongated fibers with transverse dark and light bands. The dark bands correspond to the boundaries between cells. Each fiber is made up of individual cells connected in series with each other. Cardiac muscle includes myofibrils, which are the longitudinal parallel contractile elements composed of actin and myosin filaments that are almost identical to those of the skeletal muscle. The actin and myosin filaments interdigitate and slide along each other during contraction. Contraction is caused by action potentials that propagate along or spread over the muscle fibers. The propagation of action potentials results from changes in the electrical potential across muscle cell membranes, referred to as membrane potential. The changes in the membrane potential are in turn caused by flow of sodium, potassium, and/or calcium ions across the muscle cell membranes through ion channels, which are formed by protein molecules in the cell membranes. Some types of muscle include protein structures called gap junctions through which ions flow from one muscle cell to another. Gap junctions allow the flow of ions, and hence the propagation of action potentials, directly from one cell to another. Cardiac muscle has at least two unique anatomic characteristics: a high density of calcium-sodium channels and a high density of gap junctions. These characteristics distinguish cardiac muscle from skeletal and other types of muscle.

Action potential propagates in skeletal muscle mainly via the sudden opening of fast sodium channels that allow sodium ions to enter the muscle cells. Each opening of a fast sodium channel lasts for only a few ten-thousandths of a second. In contrast, cardiac muscle includes both fast sodium channels and slow calcium-sodium channels that allow both calcium and sodium to enter the muscle cells. Each opening of a slow calcium-sodium channel lasts for several tenths of a second. This results in the long duration of contraction, which characterizes cardiac muscle.

Gap junctions in cardiac muscle fibers allow relatively free flow of ions across the cell membranes along the fiber axes. Thus, action potentials travel from one cell to another with little resistance. Cardiac muscle is a syncytium (mass of fused cells) with muscle fibers arranged in a latticework in which the fibers branch, merge, and branch again. When one cell in the syncytium becomes excited, the action potential propagates from cell to cell and spreads throughout the latticework interconnections. The heart includes two syncytiums, the atrial syncytium and the ventricular syncytium. In a normal heart, action potentials are conducted from the atrial syncytium to the ventricular syncytium through a conduction system, the A-V bundle, and the atrial syncytium contracts before the ventricular syncytium.

In one embodiment, electrical conditioning includes providing electrical stimuli such as cardiac pacing pulses to the donor cells in culture so as to cause them to contract. In another embodiment, the electrical conditioning includes providing a static electrical field to the donor cells in culture. Electrical conditioning can result in the donor cells proliferating and differentiating into cardiac muscle cells, and preferably results in cells functioning as cardiac muscle cells.

In one embodiment, electrical conditioning of donor cells results in cells with one or more characteristics of cardiac muscle cells, including a high density of calcium-sodium channels and a high density of gap junctions. Such electrical conditioning may occur in vitro and/or in vivo. Moreover, once the donor cells are implanted in the myocardium, they are subject to the pattern of contractions in the myocardium and may, if they are not cardiac muscle cells, differentiate into cardiac muscle cells. In one embodiment, the donor cells are electrically conditioned prior to implantation into the myocardium. In one embodiment, the electrical conditioning includes subjecting the donor cells to an artificially induced contraction pattern that simulates the physiological contractions of cardiac muscle cells in vivo. The contraction pattern is induced by electrical stimulation such as by cardiac pacing. In a further embodiment, the donor cells are also subjected to an electrical field stimulation that simulates the environment in the myocardium. Electrical conditioning of donor cells, including cardiac pacing and/or field stimulation, may result in an altered expression profile of the donor cells, including increased calcium-sodium channel expression and/or increased expression and/or formation of gap junctions. For instance, electrical conditioning may increase angiotensin II or VEGF expression, which in turn increases gap junction formation.

In one embodiment, pacing pulses are generated by a pacemaker or any pulse generator capable of producing the pacing pulses. The donor cells are placed in a culturing media including fluids which simulate the extracellular fluid of the myocardium. The pacing pulses are delivered to the donor cells through two electrodes placed in the culture. Parameters controlling the delivery of the cardiac pacing pulses include pacing rate, pacing voltage, and pacing pulse width, which are each selected from a physiological range to simulate the electrical activities within the myocardium. In one specific embodiment, the pacing rate is in the range of 15 to 120 beats per minute; the pacing voltage is in the range of 0.1 to 10 volts; and the pacing pulse width is in the range of 0.1 to 10 milliseconds. In one embodiment, cardiac pacing is applied to the donor cells continuously for a predetermined period of time. In one specific embodiment, the predetermined period of time is in the range of 1 to 14 days. In another embodiment, cardiac pacing is applied intermittently to the donor cells for a predetermined period that is interrupted by one or more resting (non-pacing) periods. In one specific embodiment, cardiac pacing is applied to the donor cells with a duty cycle in the range of 5% to 75% for a predetermined period that is in the range of 1 to 14 days.

In one embodiment, a static electrical field is applied to a donor cell culture. In one specific embodiment, the field strength is in the range of 1 to 100 volts per meter. In one embodiment, the electrical field is applied continuously for a predetermined period. In one specific embodiment, the predetermined period is in the range of 1 to 14 days. In another embodiment, the electrical field is applied for a predetermined period that is interrupted by one or more resting (non-stimulation) periods. In one specific embodiment, the electrical field is applied with a duty cycle of 5% to 75% for a predetermined period that is in the range of 1 to 14 days.

C. Biological Conditioning

Biological conditioning includes subjecting donor cells to exogenous agents, e.g., differentiation factors, growth factors, angiogenic proteins, survival factors, and cytokines, as well as to expression cassettes (transgenes) encoding a gene product including, but not limited to, an angiogenic protein, a growth factor, a differentiation factor, a survival factor, a cytokine, a cardiac cell-specific structural gene product, a cardiac cell-specific transcription factor, or a membrane protein, or comprising an antisense sequence, for instance, a ribozyme, or any combination thereof. The expression cassette optionally includes at least one control element such as a promoter, optionally a regulatable promoter, e.g., one which is inducible or repressible, an enhancer, or a transcription termination sequence. Preferably, the promoter and/or enhancer is one which is cell- or tissue-specific, e.g., cardiac cell-specific. For instance, the enhancer may be a muscle creatine kinase (mck) enhancer, and the promoter may be an alpha-myosin heavy chain (MyHC) or beta-MyHC promoter (see Palermo et al., Circ. Res., 78, 504 (1996)).

i. Transgenes

In one embodiment, the transgene encodes a gene product including but not limited to an angiogenic protein, e.g., a fibroblast growth factor (FGF) such as acidic-FGF, basic-FGF, and FGF-5, vascular endothelial growth factor (VEGF), e.g., VEGF₁₄₅, VEGF₁₂₁, VEGF₁₂₀, VEGF₁₆₄, VEGF₁₆₅, VEGF₁₈₉, and VEGF₂₀₆, IGF-1, TGF-beta, e.g., TGF-beta₁, leukemia inhibitory factor (LIF) alone or in combination with other cytokines, a myogenic factor, e.g., myoD, RyRZ (cardiac ryanodine receptor), Del I, myogenin, parvalbumin, Myf5, and MRF, transcription factors (GATA such as GATA-4 and dHAND/eHAND), cytokines such as cardiotrophin-1, calsequestrin, neuregulin, for instance, neuregulin 1, 2 or 3, and homeobox gene products, e.g., Csx, tinman, and the NKx family, e.g., NKx 2.5, transferrin, platelet-derived growth factor (PDGF), epidermal growth factor (EGF), adrenocorticotrophin, macrophage colony-stimulating factor, protein kinase C activators, endothelial growth factor, β2 adrenergic receptor (1 or 2), mutant G protein receptor kinase (GRK), adenylyl cylase (AC), e.g., cardiac AC such as human type II, V or VI adenyl cylase (U.S. Pat. No. 6,436,672), V2 vasopressin receptor, sarcoplasmic reticulum Ca2⁺ ATPase (SERCA2a), phospholambam, β-adrenergic receptor kinase, N-cadherin, connexin-40, connexin-42, connexin-43, contractable proteins, e.g., myosin heavy chain (MyHC), myosin light chain (MyLC), myosin binding protein C, actin, tropomyosin, troponin, e.g., troponin T, M protein, tropomodulin, myofibrillar protein, stress related protein, e.g., heat shock protein (HSP) such as HSP70i, HSP27, HSP40 or HSP60, α-1 antitrypsin, HF1-a, HF-1b, MEF2, hepatocyte growth factor (HGF), BMP-2, BMP-4, BMP-17, BMP-18, Pax7, oxytocin, oxytocin receptor, myocyte nuclear factor, Frzb (see published U.S. application 20020147329), Rb-interacting zinc finger protein (U.S. Pat. No. 6,468,985), eNOS, iNOS, serine/threonine protein phosphatase, cardiac hypertrophy factor, CT-1, α, β, γ or δ sarcoglycan, hypoxia inducible factor 1α, bcl-2, FasL, cytokine gp130 receptor, gp130, Akt, adenosine A3 receptor, angiogenin, e.g., angiogenin-1 or angiogenin-2, TNFα, dystrophin, tafazzin, desmin, lamin, troponin C, caspase inhibitors, ERK-type of MAP kinases (p42 and p44, anti-apoptosis), IL-1B, serum releasing factor, and ILGF (I and II), NGF, growth hormone, e.g., human growth hormone, or angiotensin, e.g., angiotensin II.

In another embodiment, e.g., for cells from a mammal with an inherited or acquired disorder such as one characterized by overexpression of certain endogenous genes, the transgene may comprise antisense or ribozyme sequences which substantially correspond to the reverse complement of at least a portion of the endogenous gene, and which, when expressed in a host cell, results in a decrease in the expression of the endogenous gene. Alternatively, the transgene may comprise sequences which, after homologous recombination with the endogenous gene, result in a decrease in the expression of the endogenous gene. For instance, the use of antisense vectors resulting in the decreased expression of the following gene products may be beneficial in autologous cell therapy, gene products including, but not limited to, those which induce apoptosis, e.g., Fas, Bax1 and ApoI, or a Na/Ca exchanger, or a mitogen-activated protein (MAP) kinase, Janus kinase (JAK)/signal transducer or activator of transcription, calcium/calmodulin-dependent protein phosphatase, calcineurin, carnitine palmoyl-transferase I, matrix metalloproteinase, eNOS, iNOS, serine/threonine protein phosphatase, or stress response mitogen activated protein kinase, e.g., Junk and p38-MAPK.

For purposes of the present invention, control elements, such as muscle-specific and inducible promoters, enhancers and the like, will be of particular use. Such control elements include, but are not limited to, those derived from the actin and myosin gene families, such as from the myoD gene family (Weintraub et al., Science, 251 761 (1991)); the myocyte-specific enhancer binding factor MEF-2 (Cserjesi and Olson, Mol. Cell Biol., 11, 4854 (1991)); control elements derived from the human skeletal actin gene (Muscat et al., Mol. Cell Bio., 7, 4089 (1987)) and the cardiac actin gene; muscle creatine kinase sequence elements (Johnson et al., Mol. Cell Biol., 9 3393 (1989)) and the murine creatine kinase enhancer (mCK) element; control elements derived from the skeletal fast-twitch troponin C gene, the slow-twitch cardiac troponin C gene and the slow-twitch troponin I gene; hypoxia-inducible nuclear factors (Semenza et al., Proc. Natl. Acad. Sci. USA, 88, 5680 (1991); Semenza et al., J. Biol. Chem., 269, 23757); steroid-inducible elements and promoters, such as the glucocorticoid response element (GRE) (Mader and White, Proc. Natl. Acad. Sci. USA, 90, 5603 (1993)); the fusion consensus element for RU486 induction; and elements that provide for tetracycline regulated gene expression (Dhawan et al., Somat. Cell. Mol. Genet., 21, 233 (1995); Shockett et al., Proc. Natl. Acad. Sci. USA, 92, 6522 (1995)).

Cardiac cell restricted promoters include but are not limited to promoters from the following genes: a α-myosin heavy chain gene, e.g., a ventricular α-myosin heavy chain gene, β-myosin heavy chain gene, e.g., a ventricular β-myosin heavy chain gene, myosin light chain 2v gene, e.g., a ventricular myosin light chain 2 gene, myosin light chain 2a gene, e.g., a ventricular myosin light chain 2 gene, cardiomyocyte-restricted cardiac ankyrin repeat protein (CARP) gene, cardiac α-actin gene, cardiac m2 muscarinic acetylcholine gene, ANP gene, BNP gene, cardiac troponin C gene, cardiac troponin I gene, cardiac troponin T gene, cardiac sarcoplasmic reticulum Ca-ATPase gene, skeletal α-actin gene, as well as an artificial cardiac cell-specific promoter.

Further, chamber-specific promoter promoters may also be employed, e.g., for atrial-specific expression, the quail slow myosin chain type 3 (MyHC3) or ANP promoter, may be employed. For ventricle-specific expression, the iroquois homeobox gene may be employed. Nevertheless, other promoters and/or enhancers which are not specific for cardiac cells or muscle cells, e.g., RSV promoter, may be employed in the expression cassettes and methods of the invention.

Other sources for promoters and/or enhancers are promoters and enhancers from the Csx/NKX 2.5 gene, titin gene, α-actinin gene, myomesin gene, M protein gene, cardiac troponin T gene, RyR2 gene, Cx40 gene, and Cx43 gene, as well as genes which bind Mef2, dHAND, GATA, CarG, E-box, Csx/NKX 2.5, or TGF-beta, or a combination thereof.

Preferably, the transgenic donor cells include a transgene that enhances the proliferation, engraftment, survival, differentiation and/or function of the donor cells and/or decreases, replaces or supplements (increases) the expression of endogenous genes in the donor cells. In one embodiment, the expression of the transgene is controlled by a regulatable or tissue-specific, e.g., cardiomyocyte-specific promoter. Optionally, a combination of vectors each with a different transgene can be employed.

Delivery of exogenous transgenes may be accomplished by any means, e.g., transfection with naked DNA, e.g., a vector comprising the transgene, liposomes, calcium-mediated transformation, electroporation, or transduction, e.g., using recombinant viruses. A number of transfection techniques are generally known in the art. See, e.g., Graham et al., Virology, 52, 456 (1973), Sambrook et al., Molecular Cloning: A Laboratory Manual, Cold Spring Harbor Laboratories, New York (1989), Davis et al., Basic Methods in Molecular Biology, Elsevier (1986) and Chu et al., Gene, 13, 197 (1981). Particularly suitable transfection methods include calcium phosphate co-precipitation (Graham et al., Virol., 52, 456 (1973)), direct microinjection into cultured cells (Capecchi, Cell, 22, 479 (1980)), electroporation (Shigekawa et al., BioTechniques, 6, 742 (1988)), liposome-mediated gene transfer (Mannino et al., BioTechniques, 6, 682 (1988)), lipid-mediated transduction (Felgner et al., Proc. Natl. Acad. Sci. USA, 84, 7413 (1987)), and nucleic acid delivery using high-velocity microprojectiles (Klein et al., Nature, 327, 70 (1987)). Preferred recombinant viruses to deliver exogenous transgenes to cells include recombinant lentiviruses, retroviruses, adenoviruses, adeno-associated viruses (AAV), and herpes viruses including cytomegalovirus.

In one embodiment, recombinant AAV (rAAV) is employed to deliver a transgene to donor cells. Myoblasts are transduced either while actively dividing, or as a differentiated cell culture. Differentiation is induced by placing subconfluent myoblasts in DMEM containing 2% horse serum and standard concentrations of glutamine and penicillin-streptomycin for an interval of four days prior to transduction. Verification of differentiation is by microscopic analysis to determine the presence of multinucleated myotubes in culture. Myotubes (differentiated cells) or myoblasts (dividing cells) are transduced in culture.

ii. Other Exogenous Biological Agents

In another embodiment, the exogenous biological agent includes but is not limited to an angiogenic protein, e.g., a FGF such as acidic-FGF, basic-FGF, and FGF-5, VEGF, e.g., VEGF₁₄₅, VEGF₁₂₁, VEGF₁₂₀, VEGF₁₆₄, VEGF₁₆₅, VEGF₁₈₉, and VEGF₂₀₆, IGF-1, TGF-beta, e.g., TGF-beta₁, LIF alone or in combination with other cytokines, a myogenic factor, e.g., myoD, RyRZ (cardiac ryanodine receptor), Del I, myogenin, parvalbumin, Myf5, and MRF, GATA such as GATA-4 and dHAND/eHAND, cytokines such as cardiotrophin-1, calsequestrin, neuregulin, for instance, neuregulin 1, 2 or 3, and homeobox gene products, e.g., Csx, tinman, and the NKx family, e.g., NKx 2.5, transferrin, PDGF, EGF, adrenocorticotrophin, macrophage colony-stimulating factor, protein kinase C activators, endothelial growth factor, β2 adrenergic receptor (1 or 2), mutant G protein receptor kinase (GRK), AC, e.g., cardiac AC such as human type II, V or VI adenyl cylase (U.S. Pat. No. 6,436,672), V2 vasopressin receptor, SERCA2a, phospholambam, β-adrenergic receptor kinase, N-cadherin, connexin-40, connexin-42, connexin-43, MyHC, MyLC, myosin binding protein C, actin, tropomyosin, troponin, e.g., troponin T, M protein, tropomodulin, myofibrillar protein, stress related protein, e.g., HSP such as HSP70i, HSP27, HSP40 or HSP60, α-1 antitrypsin, HF1-α, HF-1b, MEF2, HGF, BMP-2, BMP4, BMP-17, BMP-18, Pax7, oxytocin, oxytocin receptor, myocyte nuclear factor, Frzb (see published U.S. application 20020147329), Rb-interacting zinc finger protein (U.S. Pat. No. 6,468,985), eNOS, iNOS, serine/threonine protein phosphatase, cardiac hypertrophy factor, CT-1, α, β, γ or δ sarcoglycan, hypoxia inducible factor 1α, bcl-2, FasL, cytokine gp130 receptor, gp130, Akt, adenosine A3 receptor, angiogenin, e.g., angiogenin-1 or angiogenin-2, TNFα, dystrophin, tafazzin, desmin, lamin, troponin C, caspase inhibitors, ERK-type of MAP kinases (p42 and p44, anti-apoptosis), IL-1B, serum releasing factor, and ILGF (I and II), NGF, growth hormone, e.g., human growth hormone, angiotensin, e.g., angiotensin II, inotropes, norepinephrine, retinoic acid, dexamethasone, 5 azacytidine, or preconditioned media, e.g., from ES cells which contains a plurality of growth factors. Such agents may also be administered to a mammal prior to, during, or after cell therapy, or any combination thereof.

iii. Methods to Induce Differentiation

Any method may be employed to induce the differentiation of a noncardiomyocyte to a cardiomyocyte, including contacting the noncardiomyocyte cells with an exogenous agent(s) including introducing a transgene to the noncardiomyocyte cells, the expression of which in the noncardiomyocyte cell results in differentiation of the cell to a cardiomyocyte. For instance, differentiation into cardiomyocytes may be induced by treatment with a DNA demethylating agent, a factor which is expressed in the cardiogenesis region of a fetus, a factor which controls differentiation into cardiomyocytes in the cardiogenesis stage of a fetus, a culture supernatant of cells having the potential to differentiate into cardiomyocytes or a culture supernatant of cardiomyocytes differentiated from the cells. For example, MSCs can be induced to express cardiac specific genes by co-culturing MSCs with fetal, neonatal and adult rat cardiac cells, employing chemical fusigens (e.g., polyethylene glycol or sendai virus) to create heterokaryons of MSCs with fetal, neonatal and adult cardiomyocytes, incubating MSCs with extracts of mammalian hearts, including the extracellular matrix and related molecules found in heart tissue, treating MSCs with growth factors and differentiating agents, employing mechanical and/or electrical stimulation of MSCs, and mechanically and/or electrically coupling MSCs with cardiomyocytes. Similarly, endothelial cells can be induced to differentiate into cardiomyocytes when cocultured with neonatal cardiomyocytes (Condorelli et al., Proc. Natl. Acad. Sci. USA, 98, 10733 (2001). MSCs that progress towards cardiomyocytes first express proteins found in fetal cardiac tissue and then proceed to adult forms.

For ES cells, culturing those cells with hepatocyte growth factor, EGF, basic FGF, TGF-beta, BMP-2, DMSO, oxytocin, and retinoic acid can result in cultures enriched in cardiomyocytes (Boheler et al., Circ. Res., 91, 189 (2002)) (see also Xu et al., Circ. Res., 91, 501 (2002)). For example, human undifferentiated ES cells are grown on a mitotically inactivated (mitomycin C) MEF feeder layer in culture medium as described in Thomson, Science, 282, 1145 (1998). The culture medium may include 80% knockout DMEM (no-pyruvate, high-glucose formulation; Life Technologies Inc., Rockville, Md., USA) supplemented with 20% FBS (HyClone, Logan, Utah, USA), 1 mM L-glutamine, 0.1 mM mercaptoethanol, and 1% nonessential amino acid stock (all from Life Technologies Inc.). To induce differentiation, ES cells are dispersed to small clumps (three to 20 cells) using collagenase IV (Life Technologies Inc., 1 mg/ml for 20 minutes). The cells are then transferred to plastic Petri dishes (Miniplast, Ein Shemer, Israel), at a cell density of about 5×10⁶ cells in a 58-mm dish, where they are cultured in suspension for 7-10 days. During this stage, the cells aggregated to form embryoid bodies (EBs), which are then plated on 0.1% gelatin-coated culture dishes, at a density of one to five EBs in a 1.91-cm² well, and observed microscopically for the appearance of spontaneous contractions. DMSO (Sigma Chemical Co., St. Louis, Mo., USA) may be added at a concentration of 0.75% (vol/vol) to the culture medium during the 10 days of growth in suspension to enhance differentiation.

Bone marrow cells may be induced by treatment with a DNA demethylating agent, a factor which is expressed in the cardiogenesis region of a fetus, a factor which controls differentiation into cardiomyocytes in the cardiogenesis stage of a fetus, a culture supernatant of cells having the potential to differentiate into cardiomyocytes or a culture supernatant of cardiomyocytes differentiated from the cells. Any DNA demethylating agent can be used, so long as it is a compound which causes demethylation of DNA. Suitable DNA demethylating agents include demethylase which is an enzyme which specifically removes the methylation of the cytosine residue in the GpC sequence in a chromosomal DNA, 5-azacytidine (hereinafter referred to as “5-aza-C”) and DMSO (dimethyl sulfoxide). Examples of the demethylase enzymes include a demethylase disclosed in Bhattacharya et al., Nature, 397, 579 (1999). Cells having the potential to differentiate into cardiomyocytes may thus be cultured in the presence of 3 μmol/L to 10 μmol/L of 5-aza-C for 24 hours. After 5-aza-C is removed by replacing the culture supernatant with a fresh medium, the cells are cultured for further 2-3 weeks to obtain cardiomyocytes. The cardiomyocytes produced by culturing for 2-3 weeks are mainly sinus node cells, but culturing for more than 4 weeks induces differentiation into ventricular cardiomyocytes. In one embodiment, bone marrow cells are treated with 5-aza-C, BMP-2 or BMP-4 to induce differentiation.

Examples of factors which are expressed in the cardiogenesis region of a fetus and factors which act on differentiation into cardiomyocytes in the cardiogenesis stage of a fetus include cytokines, retinoic acid, adhesion molecules and transcription factors. Exemplary cytokines include PDGF, FGF-8, endothelin 1 (ET1), midkine, and BMP-4, and preferred examples of PDGF include PDGF A, PDGF B, PDGF C and the like. In one embodiment, the cytokine can be used at a concentration of 10 to 40 ng/ml. It is also possible to stimulate cardiomyogenic differentiation using an inhibitor of a cytokine which suppresses cardiomyogenic differentiation, e.g., an inhibitor of fibroblast growth factor-2 and molecules which inhibit the signal transduction of the suppressive cytokines, such as antibodies and low molecular weight compounds which neutralize cytokine activities. Examples of adhesion molecules include extracellular matrices such as gelatin, laminin, collagen, fibronectin and the like, and examples of the transcription factors include a homeobox-type transcription factor, e.g., Nkx2.5/Csx, a zinc finger-type transcription factor belonging to the GATA family, e.g., GATA4, transcription factors belonging to the myocyte enhance factor-2 (MEF-2) family, MEF-2A, MEF-2B, MEF-2C and MEF-2D, transcription factors belonging to the basic helix loop helix-type transcription factors, e.g., dHAND and eHAND, and transcription factors belonging to the family of TEA-DNA binding-type transcription factors, e.g., TEF-1, TEF-3 and TEF-5.

Cardiomyogenic differentiation may also be induced by culturing cells in a culture dish coated with an extracellular matrix obtained from spontaneously beating cardiomyocytes, co-culturing cells with spontaneously beating cardiomyocytes or adding a culture supernatant of spontaneously beating cardiomyocytes to cells.

Bone marrow cells having the potential to differentiate into cardiomyocytes may include cells which are CD117⁺ and CD140⁺. The cells which are CD117⁺ and CD140⁺ may include cells which are CD34⁺, CD117⁺ and CD140⁺, and cells which are CD34⁻, CD117⁺ and CD140⁺; as well as cells which are CD144⁺, CD34⁺, CD117⁺ and CD140⁺, cells which are CD144⁻, CD34⁺, CD117⁺ and CD140⁺, cells which are CD144⁺, CD34⁻, CD117⁺ and CD140⁺, and cells which are CD144⁻, CD34⁻, CD117⁺ and CD140⁺; cells which are CD34⁺, CD117⁺, CD14⁻, CD45⁻, CD90⁻, Flk-1⁻, CD31⁻, CD105⁻, CD144⁺, CD140⁺, CD49b⁻, CD49d⁻, CD29⁺, CD54⁻, CD102⁻, CD106⁻ and CD44⁺, cells which are CD34⁺, CD117⁺, CD14⁻, CD45⁻, CD90⁻, Flk-1⁻, CD31⁻, CD105⁻, CD144⁻, CD140⁺, CD49b⁻, CD49d⁻, CD29⁺, CD54⁻, CD102⁻, CD106⁻ and CD44⁺, cells which are CD34⁻, CD117⁺, CD14⁻, CD45⁻, CD90⁻, Flk-1⁻, CD31⁻, CD105⁻, CD144⁺, CD140⁺, CD49b⁻, CD49d⁻, CD29⁺, CD54⁻, CD102⁻, CD106⁻ and CD44⁺, and cells which are CD34⁺, CD117⁺, CD14⁻, CD45⁻, CD90⁻, Flk-1⁻, CD31, CD105⁻, CD144⁻, CD140⁺, CD49b⁻, CD49d⁻, CD29⁺, CD54⁻, CD102⁻, CD106⁻ and CD44⁺.

For hematopoietic stem cells, a growth factor which induces hematopoietic stem cells to differentiate into cardiomyocytes includes, but is not limited to, retinoic acid, stem cell factor, basic fibroblast growth factor, acidic fibroblast growth factor, endothelial cell growth factor, fibroblast growth factor-4, endothelin-1, interleukin (including, e.g., IL-2, IL-10 and Il-15), transforming growth factor alpha (TGFα), transforming growth factor beta (TGFβ), GM-CSF, IGF-1 platelet derived growth factor (PDGF), bone morphogenic factor-4, and Wnt (e.g., a member of the class Wnt5a, including e.g., Wnt11), and optionally dexamethasone, hyaluronic acid, 3,3′,5-triiodo-L-thyronine, cAMP, angiotensin II, mannose, β-mercaptoethanol, and/or a protein kinase C (PKC) inhibitor (including, e.g., sphingosine).

For fibroblast cells, EGF is a preferred agent to induce the differentiation of those cells.

iv. Exemplary Methods to Characterize the Phenotype of Donor Cells Subjected to Biological Conditioning

Methods to detect expression of a transgene in a donor cell include methods that detect transgene-specific RNA, e.g., RT-PCR, or methods that detect a gene product encoded by the transgene, e.g., via an ELISA. Examples of gene-specific assays include, for instance, those for AC (see, Salomon et al., Anal. Biochem., 58, 541 (1974); Hammond et al., Circulation, 85, 269 (1992); Hammond et al., Circulation, 8, 666 (1992)), for β-adrenergic receptor binding or content (Hammond et al., Circulation, 8, 666 (1992); Roth et al., FEBS Lett., 29, 46 (1992)), for GRK₂ and GRK₅ content (see, e.g., Ping et al., J. Clin. Invest., 95, 1271 (1995); and Roth et al., FEBS Lett, 29, 46 (1992)), and for G protein receptor kinase activity (see, Benovic, Methods Enzymology, 200, 351 (1991); Ping et al., J. Clin. Invest., 95, 1271 (1995); Ping et al., J. Clin. Invest., 95,1271 (1995); Ungerer et al., Circulation, 87, 454, (1993)).

In one embodiment, preferred donor cells are cardiomycytes, e.g., prepared from cardiac tissue or noncardiac tissue. Detection of expression of cardiomyocyte-specific proteins may be accomplished using antibodies to, for example, myosin heavy chain monoclonal antibody, e.g., MF 20 (MF20), sarcoplasmic reticulum calcium ATPase (SERCA1), e.g., mnAb 10D1, or gap junctions, e.g., using antibodies to connexin 43, as well as phospholamban, or by detecting the expression of the following genes: titin (Z-band), α-actinin, myomesin, sarcomeric myosin heavy chain, sarcomeric α-actin, cardiac tropinin T, M protein, RyR2, Cx40 and Cx 43. For the differentiation of ES cells to cardiomyocytes, the expression of the following genes may be monitored: Nkx 2.5, MEF2c, GATA 4/5/6, desmin, M-cadherin, beta1-integrin, oxytocin, oxytocin receptor, cardiac myosin heavy chain, myosin light chain 2A or 2C, cardiac tropinin I, troponin C and ANP. For the differentiation of bone marrow derived MSCs, the expression of the following genes may be monitored: beta1 and beta2 adrenergic receptors, e.g., via the response of cells to isoproterenol, or muscarinic receptors, e.g., via the response of cells to carbachol.

Atrial-like cells may be identified as cells having ion currents associated with muscarinic acetylcholine-activated K+ channels and inwardly rectifying K+ channels, but not hyperpolarization-activated pacemaker channels, while ventricular-like cells may be identified as cells having ion currents associated with inwardly rectifying K+ channels and SR ryanodine-sensitive calcium-release channels but not muscarinic acetylcholine-activated K+ channels or hyperpolarization-activated pacemaker channels. Sinus node-like cells may be identified as cells having ion currents associated with muscarinic acetylcholine-activated K+ channels and SR ryanodine-sensitive calcium release channels, and hyperpolarization-activated pacemaker channels but not inwardly rectifying K+ channels.

In one embodiment, donor cells subjected to in vitro biological conditioning spontaneously contract or beat.

Compositions, Dosages and Routes of Administration of the Donor Cells

Compositions of the invention comprise donor cells, including cells from different sources, and optionally agents that enhance donor cell engraftment, survival, proliferation and/or differentiation, enhance cardiac function or stimulate angiogenesis. The cells to be administered may be a population of individual cells or cells grown in culture so as to form a two dimensional or three dimensional structure. The number of cells to be administered will be an amount which results in a beneficial effect to the recipient. For example, from 10² to 10¹⁰, e.g., from 10³ to 10⁹, 10⁴ to 10⁸, or 10⁵ to 10⁷, cells can be administered to, e.g., injected, the region of interest, for instance, infarcted and tissue surrounding infarcted tissue. Agents which may enhance cardiac function or stimulate angiogenesis include but are not limited to pyruvate, catecholamine stimulating agents, fibroblast growth factor, e.g., basic fibroblast growth factor, acidic fibroblast growth factor, fibroblast growth factor-4 and fibroblast growth factor-5, epidermal growth factor, platelet-derived growth factor, vascular endothelial growth factor (e.g., VEGF₁₂₁, VEGF₁₄₅, VEGF₁₆₅, VEGF₁₈₉ or VEGF₂₀₆), tissue growth factors and the like. Such agents may optionally be present in the compositions of the invention or administered separately.

The cells are administered during a prophylactic, diagnostic or therapeutic vascular procedure or an invasive or minimally invasive surgical procedure. In one embodiment, the cells are administered post-myocardial infarction, within hours, e.g., 1 to 12 hours, to days, e.g., 1 to 2 days, and up to one or more weeks after myocardial infarction. Preferably, the administration of donor cells is prior to scar formation. The cells may be administered intravenously, transvenously, intramyocardially or by any other convenient route, and delivered by a needle, catheter, e.g., a catheter which includes an injection needle or infusion port, or other suitable device. Some exemplary delivery apparatus and methods include, but are not limited to, the teachings provided herein.

In one embodiment, once administered, the donor cells develop functional connections with adjacent cells, membrane channels with adjacent cells, including viable cells in the recipient, and, if not already differentiated, differentiate to myocardial cells.

### EXAMPLE OF ELECTRICAL THERAPY OF CARDIAC TISSUE

Following cell therapy, the identified region of tissue to be treated is subjected to electrical therapy at 150. In the example of cardiac tissue, electric current is imposed across or adjacent to the damaged tissue. In one embodiment a pacemaker with implanted catheter leads is employed to provide the appropriate pacing stimulation to the identified region of tissue. In varying embodiments, one or more electrodes serve to apply an electric field over portions of the identified tissue region. In implanted pacemaker applications the pacemaker housing may serve as an electrode.

In one embodiment, the pacemaker is programmed to perform VDD pacing using an atrioventricular delay which is relatively short when compared to the intrinsic atrioventricular interval. In such embodiments, the electrical pace wavefront is near the infarcted region very early in the cardiac cycle so as to electrophysiologically capture and mechanically unload the identified region with the pacing stimulus. The VDD mode of the pacemaker allows the heart to maintain a rate near to that of a normal sinus rhythm, providing better control of the activation pattern; the ventricles are pre-excited without advancing the pacing rate unnecessarily. In this way, the depolarization wavefront fuses with the paced complex, resulting in the most intrinsic activation of the ventricles, yet providing for the pre-excitement of the damaged tissue region. In another embodiment, the pacemaker is programmed to perform DDD pacing using an atrioventricular delay which is relatively short when compared to the intrinsic atrioventricular interval (measured when at least the ventricular beat is intrinsic). The DDD mode of the pacemaker forces the heart to beat in a normal or desired rate when the heart fails to maintain the normal sinus rhythm. The VDD and DDD modes each includes a biventricular version where both the right ventricle (RV) and the left ventricle (LV) are paced, at same or different atrioventricular delays. Other pacing modes are possible, and those provided here are not intended in an exhaustive or exclusive sense.

In varying embodiments and combinations, the electrical therapy includes different programming modes for use with a particular cell therapy. In one embodiment, electrical therapy is invoked during periods of relative inactivity such as are common during nocturnal sleep to condition the cardiac tissue and improve cell engraftment. In one embodiment, electrical therapy is invoked based on physical activity of the patient during which heart wall stress is reduced via electrical pre-excitation. Such physical activity may be measured by detection of accelerometer data. In one embodiment, the electrical therapy is invoked for certain times of day or during specifically programmed, recurring patterns of intrinsic (M beats) and paced beats (N beats) in a ratio of M:N. In embodiments featuring programmable microprocessors, the time of day is downloaded to the microprocessor upon programming and therapy is programmably selectable. In varying embodiments and combinations, electrical therapy is delivered upon preselected sensor inputs. For example, electrical therapy is invoked (continuous or M:N patterns) upon detected patient activity. In one embodiment, electrical therapy is invoked upon detection of patient stress. In one embodiment, electrical therapy is invoked upon detection of patient metabolic high stress in the heart, such as in sleep, where ventricles are distended and filling better. In one embodiment internal pressure is measured to determine local stress. Different sensors may be employed to determine conditions for delivery of electrical therapy.

Additional programming modes are contemplated by the present description. For example, in one embodiment a variable programming mode incorporates traditional electrical pacing interspersed with specialized cell therapy pacing cycles. In one embodiment, such pacing is used to provide complementary pacing therapies to a patient's heart to provide multiple benefits. In one embodiment, the varying pacing is applied using a duty-cycle approach. For example, a ratio of pacing of a first type to a pacing of a second type is programmed into the implantable device to provide a plurality of pacing therapies to a patient. This provides a new pacing mode where the programmability of duty cycle affords electrical therapy that complements at least one other pacing therapy and the administered cell therapy.

Another pacing variation provides a dynamically changing atrioventricular delay. In one exemplary embodiment, an atrioventricular delay is increased over a predetermined time period. For one example, an atrioventricular delay is lengthened by approximately one (1) millisecond each day over a predetermined time, such as three (3) months. In one embodiment, the atrioventricular delay is lengthened by 10 milliseconds over a predetermined amount of time, such as 2 months. In such embodiments, incremental increase in atrioventricular delay results in progressively loading a cardiac region, based on location of the electrodes. Similar but opposite effects might be obtained by progressively shortening the atrioventricular delay. Certain areas of the myocardium might be progressively unloaded, resulting in desired phenotypical changes at the chamber, tissue and cell levels.

Other embodiments and combinations are possible without departing from the scope of the present therapy system. The foregoing examples are intended to demonstrate some varying embodiments of the present therapy system, and are not intended in an exclusive or exhaustive sense.

In one embodiment, the pacing lead is positioned as close as possible to the site of engraftment. Positioning is performed using electrophysiology (e.g., ECG), echocardiographic mapping, or catheter based voltage mapping of the heart. Other location methods are possible without departing from the scope of the present teachings.

Lead placement is possible using epicardial leads implanted with minimal thorocotomy, and/or catheter leads. Treatment of the left ventricular region is possible using leads positioned in the coronary venous structures.

It is understood that a plurality of infarcted tissue regions may be treated using multiple cell and electrical therapy treatments.

Non-human animal models, e.g., rodent, lapine, canine or swine models, may be employed to determine pacing and cellular parameters useful to inhibit or treat a particular indication or condition. See, e.g., Jain et al., supra; Suzuki et al., supra; Pouleur et al., Eur. J. Clin. Investig., 13, 331 (1983); Hammond, J. Clin. Res., 92, 2644 (1993); Taylor et al., Proc. Assoc. Am. Phys., 109, 245 (1997); and Roth et al., J. Clin. Res., 91, 939 (1993)). For an animal model of myocardial infarction, efficacious pacing and cell therapy results in improvement in cardiac function, e.g., increased maximum exercise capacity, contractile performance, and propagation velocity, decreased deleterious remodeling, decreased post-scar expansion, decreased apoptosis, increased angiogenesis, and increased donor cell engraftment, survival, proliferation, and function. Donor cell function can be determined using biochemical markers, e.g., myotube formation in grafted donor cells, the presence and/or levels of α-actinin, titin, myomesin, sarcomeric myosin heavy chain, α-actin and the like, and gap junction proteins (see Pimentel et al., Circulation Res, 90, 671 (2002)), as well as by improvements in global and regional cardiac function in recipients of donor cells. In ex vivo models, systolic and diastolic pressure-volume relations can be used to determine the efficacy of a particular therapy.

### EXAMPLE CARDIAC FUNCTION MANAGEMENT DEVICE

FIG. 3 shows a pacemaker performing the electrical therapy described herein. As used herein, the term pacemaker should be taken to mean any cardiac rhythm management device for pacing the heart and includes implantable pacemakers, external pacemakers, and implantable cardiac defibrillator/converters having a pacing functionality. A block diagram of a cardiac pacemaker having two ventricular pacing channels is shown in FIG. 3. Microprocessor 310 communicates with a memory 312 via a bidirectional data bus. In varying embodiments memory 312 comprises a ROM or RAM for program storage and a RAM for data storage. In one embodiment, the control unit includes dedicated circuitry either instead of, or in addition to, the programmed microprocessor for controlling the operation of the device. In one embodiment, the pacemaker employs a programmable microprocessor to implement the logic and timing functions for operating the pacemaker in accordance with a specified pacing mode and pacing parameters as well as for performing the data acquisition functions. A telemetry interface 340 is also provided for communicating with an external programmer. Such an external programmer may be used to change the pacing mode, adjust operating parameters, receive data stored by the device, and issue commands that affect the operation of the pacemaker. Such an interface also provides communications with advanced patient management devices, such as portable computers, PDA's, and other wireless devices as described herein and provided by the documents incorporated herein.

In embodiments incorporating physical motion detection for application of therapy the pacemaker includes sensors to detect exercise. For example, accelerometers and minute ventilation sensors may be incorporated for these purposes. Some embodiments may incorporate time of day for application of therapy. Such embodiments may include timing modules and may update them using information from a programmer or other wireless device.

The pacemaker has atrial sensing/stimulation channels comprising electrode 334, lead 333, sensing amplifier/filter 331, pulse generator 332, and an atrial channel interface 330 which communicates bidirectionally with a port of microprocessor 10. The device also has two ventricular sensing/stimulation channels that include electrodes 324A-B, leads 323A-B, sensing amplifiers 321A-B, pulse generators 322A-B, and ventricular channel interfaces 320A-B where “A” designates one ventricular channel and “B” designates the other. For each channel, the same lead and electrode are used for both sensing (i.e., detecting P-waves and R-waves) and stimulation. The ventricular electrodes could be disposed in each of the ventricles for biventricular pacing or in only one ventricle for multi-site pacing of that ventricle. The channel interfaces 320A-B and 330 include analog-to-digital converters for digitizing sensing signal inputs from the sensing amplifiers and registers which can be written to by the microprocessor in order to output stimulation pulses, change the stimulation pulse amplitude, and adjust the gain and threshold values for the sensing amplifiers. After digitization of the sensed signals by the channel interfaces, the signal samples can be processed in the digital domain by algorithms executed by the microprocessor in order perform further filtering. The detection of R wave and P wave peaks for timing purposes can also be performed digitally. Alternatively, a standard peak detection circuit could be used.

In one embodiment, the lead system includes endocardial leads, although other types of leads, such as epicardial leads, could also be used within the scope of the present teachings. In one embodiment, a first ventricular lead system is adapted for placement in a first cardiac region of the heart. In one example, the first cardiac region of the heart is within the coronary sinus and/or the great cardiac vein of the heart adjacent to the left ventricle. In one embodiment, the first lead system includes a number of electrodes and electrical contacts. A tip electrode is located at, or near, the distal end of the first lead system, and connects electrically to terminal through a conductor provided within the first lead system. The first lead system also includes a proximal electrode which is spaced proximal the tip electrode. In one embodiment, the proximal electrode is spaced proximal the tip electrode for placement adjacent to the left ventricle of the heart. The proximal electrode is electrically connected to terminal through an internal conductor within the first lead system. The proximal electrode can be of either an annular or a semi-annular construction, encircling or semi-encircling the peripheral surface of the first lead system.

The pacemaker further includes a second ventricular lead system. In one embodiment, the second lead system is an endocardial lead, although other types of leads, such as epicardial leads, could be used within the scope of the present teachings. The second ventricular lead system is adapted for placement within a second cardiac region of the heart. In one example, the second cardiac region of the heart is the right ventricle of the heart. In one embodiment, the second lead system includes a number of electrodes and electrical contacts. For example, in one embodiment, a tip electrode is located at, or near, the distal end of the second lead system, and connects electrically through a conductor provided in the lead, for connection to terminal. The second lead system further optionally includes a first defibrillation coil electrode spaced proximal to the distal end for placement in the right ventricle. The first defibrillation coil electrode is electrically connected to both terminals and through internal conductors within the body of the second lead system. The second lead system also optionally includes a second defibrillation coil electrode, which is spaced apart and proximal from the distal end of the second lead system such that the second defibrillation coil electrode is positioned within the right atrium or major vein leading to the right atrium of the heart. The second defibrillation coil electrode is electrically connected to terminal through an internal conductor within the body of the second lead system.

In varying embodiments, the system includes multiple atrial electrodes and optionally includes the defibrillation components. The configuration and placement of electrodes may vary without departing from the scope of the present teachings.

In one embodiment, the pacemaker is a programmable microprocessor-based system, with a microprocessor and memory, which contains parameters for various pacing and sensing modes. Pacing modes include, but are not limited to, normal pacing, overdrive or burst pacing, and pacing for prevention of ventricular tachyarrhythmias. The system also includes means for adjusting atrioventricular delay. The microprocessor further includes means for communicating with an internal controller, in the form of an RF receiver/transmitter. This includes an antenna, whereby it may receive and transmit signals to and from an external controller. In this manner, programming commands or instructions can be transferred to the microprocessor after implant. In one embodiment operating data is stored in memory during operation. This data may be transferred to the external controller for medical analysis.

In one embodiment, pacing pulses are controlled by the microprocessor to carry out a coordinated pacing scheme at the two ventricular pacing locations. Pacing modes include, but are not limited to, normal sinus rhythm pacing modes, overdrive or burst pacing modes for treating ventricular tachyarrhythmia, and/or pacing regimens for preventing the onset of a ventricular tachyarrhythmia. Additional advantages for providing pacing from the two ventricular pacing locations include the ability for either one of the two pacing systems to serve as a back-up pacing system and location for the other in the event that one pacing system were to fail.

Atrial sensing circuit is coupled by an atrial lead to a heart for receiving, sensing, and/or detecting electrical atrial heart signals. Such atrial heart signals include atrial activations (also referred to as atrial depolarizations or P-waves), which correspond to atrial contractions. Such atrial heart signals include normal atrial rhythms, and abnormal atrial rhythms including atrial tachyarrhythmias, such as atrial fibrillation, and other atrial activity. An atrial sensing circuit provides one or more signals to controller to indicate, among other things, the presence of sensed intrinsic atrial heart contractions.

An atrial therapy circuit provides atrial pacing therapy, as appropriate, to electrodes located at or near one of the atria of the heart for obtaining resulting evoked atrial depolarizations. In one embodiment, the atrial therapy circuit also provides cardioversion/defibrillation therapy, as appropriate, to electrodes located at or near one of the atria of the heart, for terminating atrial fibrillation and/or other atrial tachyarrhythmias.

Although FIG. 3 shows an implanted cardiac rhythm management device, it is understood that the teachings may be used with devices other than cardiac rhythm management devices. The teachings are also applicable to non-mammalian heart therapies. Those skilled in the art, upon reading and understanding the present description, shall appreciate other uses and variations within the scope of the present teachings.

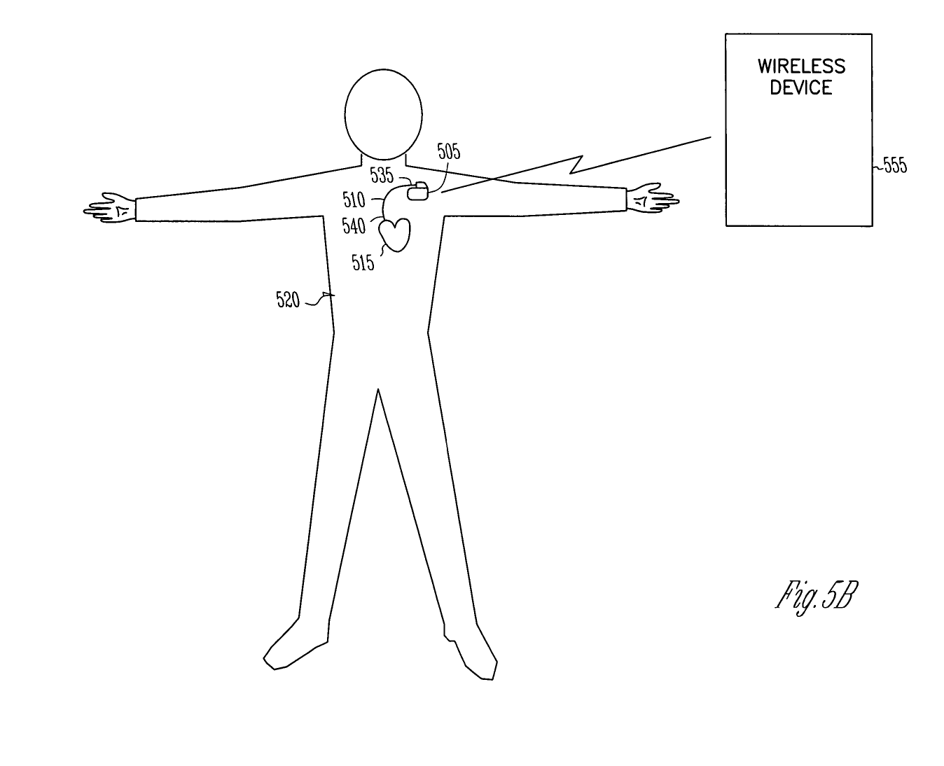

FIG. 4 shows one example of administration of cell therapy and electrical therapy to a region of cardiac tissue subject to myocardial infarction. The heart 402 includes a left ventricle 404 which has tissue injured by a myocardial infarction in an affected region 400. Affected region 400 is determined by methods including those described herein. Cell therapy 406 is preferably administered in close proximity to, e.g., transvenously, transarterially, intramyocardially or in adjacent non-infarcted tissue, and/or directly to the affected region 400 and electrical therapy is applied using a programmable pulse generator 408 and lead 410.

The electrical therapy includes pacing in vivo preferably near infracted or hybemating myocardium and including sites targeted for cell therapy to enhance the engraftment, survival, proliferation, and/or function, and optionally the differentiation, of the cells. The pacing may be applied to lessen local stress and strain that might otherwise inhibit the successful engraftment of donor cells including the successful formation of gap junctions between donor cells and noninfarcted recipient myocardial cells. Such therapy thus affects both mechanical and electrical connections to neighboring cells of the native myocardium. In particular, pacing at or near such sites may enhance development of new gap junctions which may be needed for coordinating the function of the donor cells with that of the native myocardium. The therapy also operates to control metabolic demands at the site of targeted cell therapy to increase donor cell viability. Another benefit is that electrical stimulation of myocytes promotes release of factors that encourage angiogenesis. In one embodiment, electrical therapy improves the local environment in damaged cardiac tissue, e.g., by improving pump efficiency, oxygen consumption, and/or mechanical synchrony, decreasing metabolic load and/or stress, and/or reorienting stress-strain patterns. In one embodiment, preconditioning of cells cultured in vitro, e.g., with drugs or other chemical agents, and/or transgene expression, and/or electrical stimulation and/or mechanical stimulation, may benefit in vivo engraftment, survival, proliferation, differentiation and/or functioning of the cells.

In vivo left ventricle pacing controls local stress by managing atrioventricular delay, RV-LV offset (e.g., applying an interventricular delay between RV and LV pacing pulse deliveries, or two independent atrioventricular delays for RV and LV pacing pulse deliveries), stimulation site alternation, heart rate, and pacing waveform parameters. The LV stimulus also promotes donor cell engraftment, survival, proliferation, differentiation and/or functioning in vivo and is controllable based on pacing waveform, rate, and site.

In one embodiment, the pacemaker is programmed to perform VDD pacing using an atrioventricular delay which is relatively short when compared to the intrinsic atrioventricular interval. In another embodiment, the pacemaker is programmed to perform DDD pacing using an atrioventricular delay which is relatively short when compared to the intrinsic atrioventricular interval (measured when at least the ventricular beat is intrinsic). Other electrical therapies are possible given the teachings herein. For example, it is possible that the affected region is pre-treated to strengthen the region before injection of cell therapy. Upon reading and understanding the teachings provided herein, those skilled in the art will understand other electrical therapies are possible without departing from the scope of the present teachings.

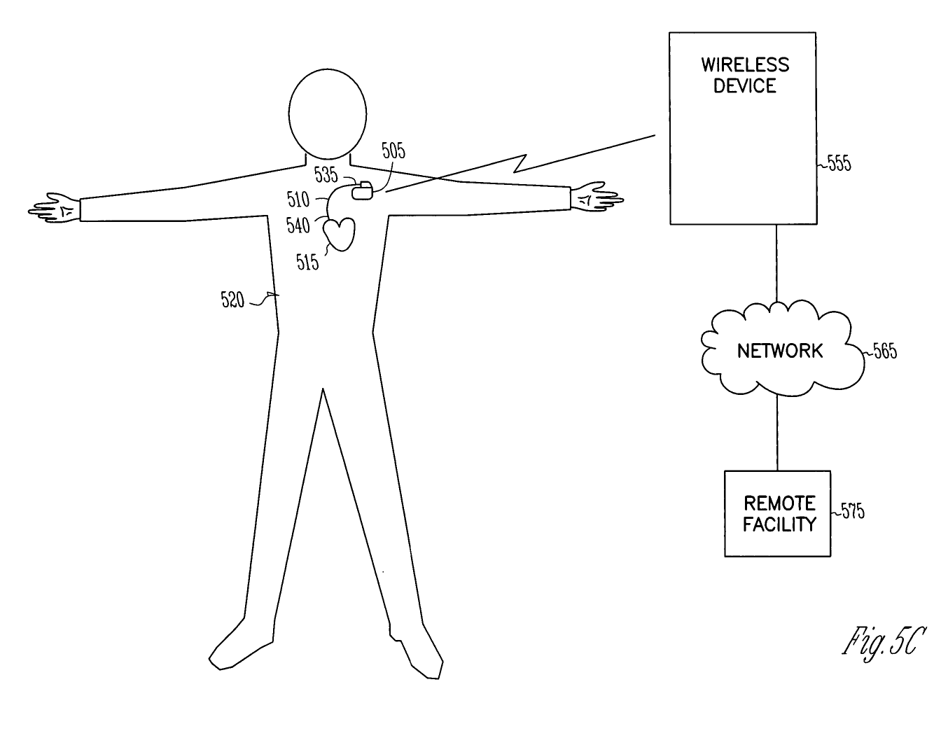

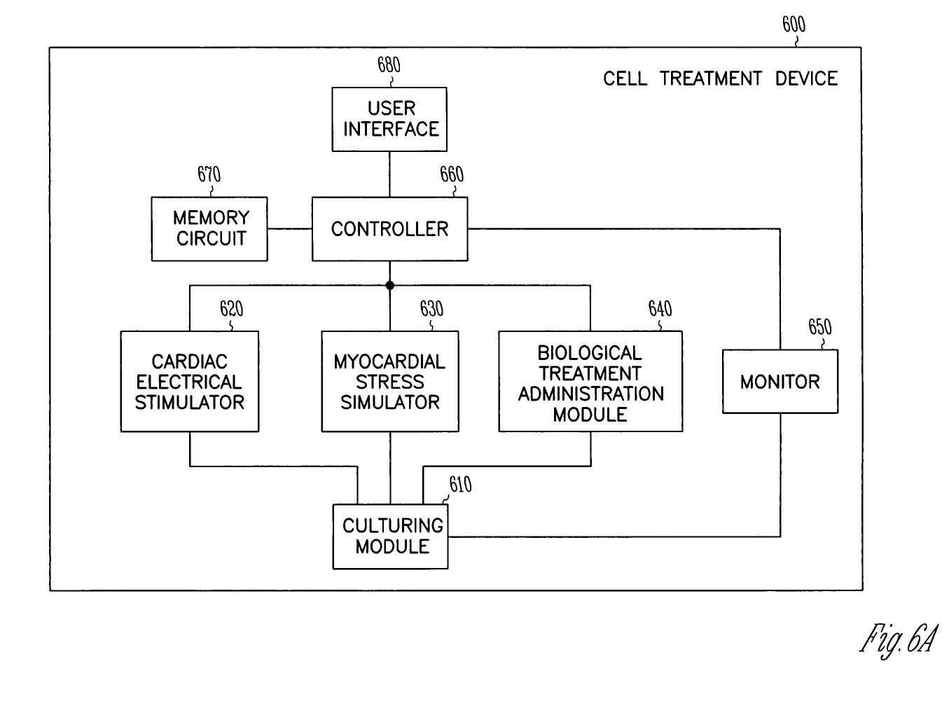

FIG. 5A is a schematic drawing illustrating, by way of example, but not by way of limitation, one embodiment of portions of a cardiac rhythm management system 500 and an environment in which it is used. System 500 includes an implantable cardiac rhythm management device 505, also referred to as an electronics unit, which is coupled by an intravascular endocardial lead 510, or other lead, to a heart 515 of patient 520. Implantable cardiac rhythm management device 505 includes a pacemaker. System 500 also includes an external programmer 525 providing wireless communication with implantable cardiac rhythm management device 505 using a telemetry device 530. Lead 510 includes a proximal end 535, which is coupled to implantable cardiac rhythm management device 505, and a distal end 540, which is coupled to one or more portions of heart 515. Although FIG. 5A shows a human with an implanted cardiac rhythm management device, it is understood that the teachings may be used with devices other than cardiac rhythm management devices. The teachings are also applicable to non-mammalian heart therapies. Those skilled in the art, upon reading and understanding the present description, shall appreciate other uses and variations within the scope of the present teachings.

FIG. 5B is a diagram showing a wireless device in communications with an implanted device for management of the implanted device and therapy according to one embodiment of the present invention. In one embodiment, wireless device 555 is used to conduct communications with implantable cardiac rhythm management device 505. In one application, wireless device 555 is a personal digital assistant (PDA). In one embodiment, wireless device 555 is a computer with wireless interface. In one embodiment, wireless device 555 is a cellular phone. The communications between implantable cardiac rhythm management device 505 and wireless device 555 can be used for coordinating operations and therapies of the pacemaker and/or to communicate device operations and physiological data to another site in communications with the wireless device 555. FIG. SC shows one example of communications where a network 565 is in contact with wireless device 555. The connection between wireless device 555 and network 565 can be either wired or wireless. In one embodiment, network 565 is the Internet. Remote facility 575 is a medical facility or location which a doctor or health care provider can access data from implantable cardiac rhythm management device 505. Alternatively, data and/or instructions can be transmitted from the remote facility 575 to the wireless device 555 and/or the implantable cardiac rhythm management device 505. Alternatively, instructions and data can be transferred bidirectionally between the remote facility, wireless device, and/or implantable cardiac rhythm management device 505.

The network is a communication system that interconnects a number of computer processing units when those units are some distance away from one another, but within the same contiguous property to allow private communications facilities to be installed. The network may also include the facility to allow multiple compute processors to communicate with each other when some or all of those processors are within the same enclosure and connected by a common back plane.

Connections with a remote facility and wireless device are useful for advanced patient management. Some exemplary apparatus and methods for patient management include, but are not limited to, the teachings provided in the patent application entitled: Method and Apparatus for Establishing Context Among Events and Optimizing Implanted Medical Device Performance, U.S. patent application Ser. No. 10/093,353, filed Mar. 6, 2002, which is incorporated by reference in its entirety.

### EXAMPLE OF IN VITRO CELL TREATMENT DEVICE

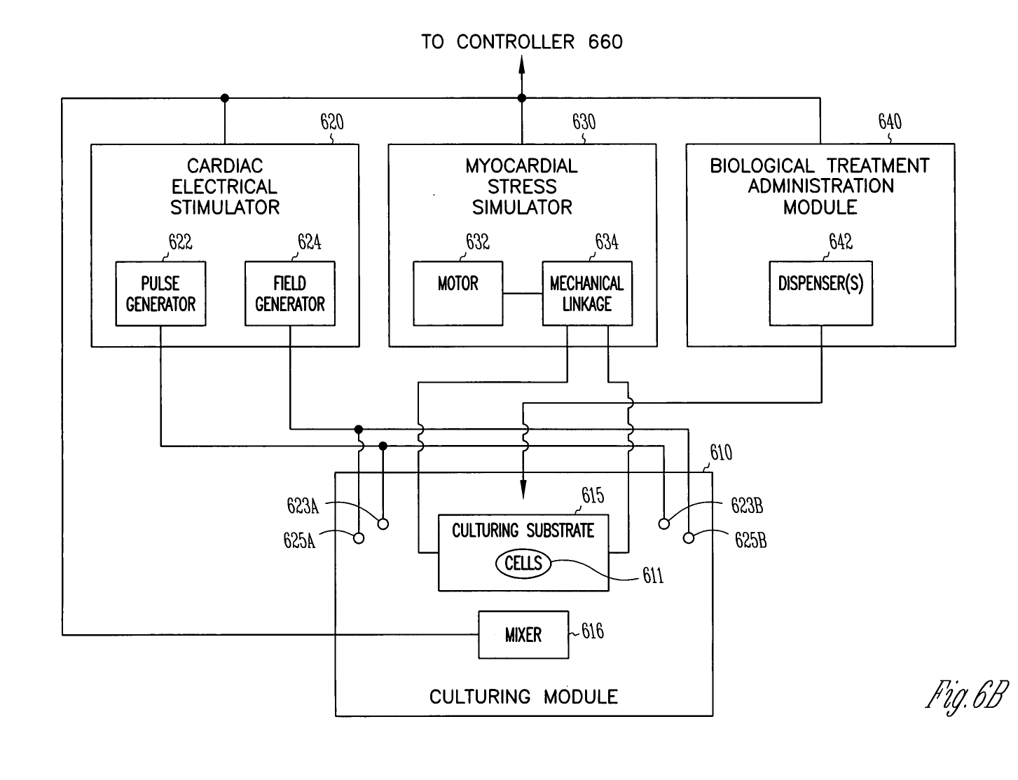

FIG. 6A shows a block diagram of a cell treatment device 600 performing the in vitro conditioning (stimulation) described above. Cell treatment device 600 includes functional modules treating donor cells in vitro by applying one or more of electrical, mechanical, and biological stimuli. Donor cells are placed in a culturing module 610 containing a culturing medium. A cardiac electrical stimulator 620, a myocardial stress simulator 630, and a biological treatment administration module 640 are connected to culturing module 610 to allow delivery of the stimuli to the donor cells. A monitor 650 provides means for observing the donor cells in culturing module 610. A controller 660 is connected to cardiac electrical stimulator 620, myocardial stress simulator 630, and biological treatment administration module 640 to control the timing and magnitude for the delivery of the electrical, mechanical, and/or biological stimuli. A memory circuit 670 connected to controller 660 provides a medium for storing instructions for an automated process of in vitro conditioning of donor cells. A user interface 680 allow a user to control and monitor the progress of the in vitro conditioning. FIG. 6B shows additional details of portions of cell treatment device 600.

Culturing module 610 includes a container to host the donor cells and the culturing medium. In one embodiment, donor cells 611 are placed on a culturing substrate 615 in culturing module 610. Culturing substrate 615 is deformable in two or more directions and can be cyclically stretched and relaxed. In one embodiment, culturing substrate is a strip made of silicone. When culturing substrate 615 is stretched and relaxed in two or more directions, donor cells are stretched and relaxed with it. In one embodiment, culturing module 610 include a mixer 616 for creating and maintaining a homogeneous culturing medium. In one embodiment, the culturing medium includes chemical and/or biochemical agents introduced as biological stimulants during the biological conditioning. When being turned on, mixer 616 shakes and/or stirs the culturing medium.

Cardiac electrical stimulator 620 provides for the in vitro electrical conditioning of donor cells, as described in this document. Cardiac electrical stimulator 620 creates cardiac electrical conditions in the culturing medium, and thus exposes donor cells 611 to such conditions. The cardiac electrical conditions simulate the electrical conditions in the myocardium which result in contraction of the heart. In one embodiment, cardiac electrical stimulator 620 includes an electrical pulse generator 622 and an electrical field generator 624. Two electrodes 623A and 623B connected to pulse generator 622 are disposed in the culturing medium to allow delivery of electrical energy in a form of current or voltage pulses to donor cells 611. Two electrodes 625A and 625B connected to field generator 624 are disposed in the culturing medium to create an electrical field across donor cells 611. In one embodiment, electrodes 623A and 625A are physically integrated, and electrodes 623B and 625B are physically integrated. Delivery of the electrical energy activates donor cells 611, causing them to contract like cardiac muscle cells in the myocardium. In one embodiment, pulse generator 622 is a pacemaker. Field generator 624 includes a dc voltage generator that creates an electrical field by applying a voltage over a known distance between electrodes 625A and 625B.

Myocardial stress simulator 630 provides for the in vitro mechanical conditioning of donor cells, as described above in this document. Myocardial stress simulator 630 creates a mechanical stress upon donor cells 611. The mechanical stress simulates the tension applied upon cardiac muscle cells in the myocardium. The tension results from mechanical forces created by the cyclical changes in heart volume and intracardiac blood pressure. In one embodiment, myocardial stress simulator 630 includes a variable speed motor 632 and a mechanical linkage 634. Mechanical linkage 634 provides for the interface between motor 632 and culturing substrate 615 allowing a controlled motion of motor 632 to create a calibrated cyclic mechanical tension on culturing substrate 615. In one embodiment, mechanical linkage 634 allows the culturing substrate 615 to be cyclically stretched and relaxed in two or more directions without vibration and hesitation. As described above, one example of such a mechanical stimulator is given in Terracio et al., In Vitro Cellular & Developmental Biology, 24(1), 53-58, 1988.

Biological treatment administration module 640 provides for the in vitro biological conditioning of the donor cells, as described above in this document. Biological treatment administration module 640 introduces exogenous agents to the culturing medium, causing one or more biological reactions in the donor cells, and hence one or more changes in the biological properties of the donor cells. In one embodiment, biological treatment administration module 640 includes one or more chemical dispenser(s) 642 to allow controlled release of one or more chemical or biochemical agents into the culturing medium. In one embodiment, biological treatment administration module 640 includes an array of dispensers each controlled for releasing a predetermined amount of chemical or biochemical agent(s) into the culturing medium at one or more predetermined times.

User interface 680 includes a user input device to accept commands from the user and a presentation device to inform the user of the status and the progress of the in vitro conditioning. In one embodiment, the user input device includes a keyboard. The presentation device includes a display screen. In another embodiment, the presentation device includes a printer. In yet another embodiment, at least portions of the user input device and the presentation devices are integrated as an interactive screen.

In one embodiment, monitor 650 includes a microscope aimed at donor cells 611 on substrate 615 and connected to controller 660. Controller 660 processes the image of donor cell 611 or a portion of it and present the image through user interface 680. In one embodiment, the image of donor cells are displayed on the display screen of user interface 680.

Controller 660 controls the magnitude (or intensity) of each of the electrical, mechanical, and biological stimuli and coordinates the timing for delivering those stimuli. The magnitude of the electrical stimulation includes, but is not limited to, a pacing voltage and a pacing pulse width. The magnitude of the mechanical stimulation includes, but is not limited to, a frequency and a degree of donor cell deformation such as an extension of cell length by a predetermined percentage of the original cell length. The magnitude of the biological stimulation includes, but is not limited to, a volume and a concentration of each chemical and/or biochemical agent. The timing includes, but is not limited to, starting times and durations that control the deliveries of all the electrical, mechanical, and/or biological stimuli in a predetermined sequence of stimulation, i.e., the complete in vitro conditioning. In one embodiment, a user controls the complete in vitro conditioning process, or portions of the process, by entering parameters defining the magnitude and timing of each stimulus and giving a command to deliver one stimulus or one sequence of stimuli through user interface 680. In another embodiment, an instruction set defining a predetermined sequence of electrical, mechanical, and/or biological stimuli, including the required magnitude and timing, is stored in memory circuit 670. Controller 660 controls cardiac electrical stimulator 620, myocardial stress simulator 630, and biological treatment administration module 640 by automatically executing the instruction set. In a further embodiment, controller 660 allows the user to adjust parameters in the instruction set during the in vitro conditioning in response to cell reactions observed through monitor 650.

### COMBINED CELL AND ELECTRICAL THERAPY EXAMPLE

In one embodiment, skeletal muscle cells are obtained from a patient who recently, e.g., within the previous 1 to 7 days, suffered a myocardial infarction. The skeletal muscle cells are cultured and conditioned in vitro, e.g., so as to expand the population, or may be employed in the absence of culturing and conditioning. Prior to cell therapy, the damaged tissue in the patient is located by conventional means, e.g., an electrocardiogram or MRI. The autologous donor skeletal muscle cells, prior to administration to the damaged tissue, may be optionally subjected to washing to remove non-cellular components, i.e., components which are not intact cells including components in tissue culture media, and introduced to the damaged tissue in a physiologically compatible carrier (vehicle), e.g., an aqueous, semi-solid or solid vehicle. In one embodiment, approximately 10² to 10¹⁰ donor skeletal muscle cells are administered via a catheter, which includes an injection needle, plurality of needles, or infusion port, positioned at or near the damaged tissue. A biocompatible (e.g., biodegradable) marker may be administered with the skeletal muscle cells so as to monitor the site(s) of administration of the donor cells and, optionally, later identify the treated region. Once administered, the donor cells develop functional connections with adjacent viable cells, and membrane channels with adjacent viable cells.

In one embodiment, the area including the damaged tissue and donor cells in the patient are then subjected to electric conditioning, such as pacing-level electrical stimulation, using a pulse generator with properly positioned electrodes, which in combination with cell therapy results in an improvement in global and regional cardiac function in the patient. A pacing regimen is provided where the pacemaker is programmed to perform VDD pacing using an atrioventricular delay which is relatively short when compared to the intrinsic atrioventricular interval.

In General

Although the present therapy is described in the example of cardiac therapy, it is understood that many other applications are possible. Such teachings may be applied to in vitro and in vivo treatment of other organs and blood vessel growth.

It is to be understood that the above description is intended to be illustrative, and not restrictive. Other embodiments will be apparent to those of skill in the art upon reviewing and understanding the above description. The scope of the invention should, therefore, be determined with reference to the appended claims, along with the full scope of equivalents to which such claims are entitled.

What is claimed is:

1. An apparatus adapted for in vitro conditioning of cells prior to administration of the cells into tissue in a cell therapy, the apparatus comprising:
a culturing module to host the cells and a culturing medium;
a cardiac electrical stimulator coupled to the culturing module;
a myocardial stress simulator coupled to the culturing module;
a biological treatment administration module coupled to the culturing module; and
a controller coupled to the cardiac electrical stimulator, the myocardial stress simulator, and the biological treatment administration module, the controller adapted to control a delivery of one or more stimuli from one or more of the cardiac electrical stimulator, the myocardial stress simulator, and the biological treatment administration module.

2. The apparatus of claim 1, further comprising two or more electrodes, connected to the cardiac electrical stimulator and disposed in the culturing medium, to allow delivery of at least one electrical stimulus to the cells.

3. The apparatus of claim 2, wherein the electrical stimulator comprises a pacemaker.

4. The apparatus of claim 3, wherein the cardiac electrical stimulator comprises an electric field generator.

5. The apparatus of claim 1, wherein the culturing module comprises a deformable culturing substrate allowing the cells to be plated thereon.

6. The apparatus of claim 5, wherein the deformable culturing substrate is made of silicone.

7. The apparatus of claim 6, wherein the myocardial stress simulator comprises a variable speed motor and a mechanical linkage coupled between the variable speed motor and the deformable culturing substrate, the variable speed motor and the mechanical linkage adapted to create a calibrated cyclic mechanical tension upon the deformable culturing substrate.

8. The apparatus of claim 1, wherein the biological treatment administration module comprises one or more chemical dispensers adapted to release one or more biological stimulation agents into the culturing medium.

9. The apparatus of claim 8, wherein the culturing module comprises a mixer adapted to create and maintain a homogeneous culturing medium.

10. The apparatus of claim 1, further comprising a user interface coupled to the controller, the user interface including a use input accepting commands.

11. The apparatus of claim 10, wherein the controller comprises a memory circuit storing an instruction for an automated delivery of a sequence of one or more of electrical, mechanical, and biological stimuli.

12. The apparatus of claim 11, wherein the user interface comprises a display screen.

13. The apparatus of claim 12, further comprising a monitor coupled to the culturing module, the monitor adapted for observation of the cells in the culturing module.

14. The apparatus of claim 13, wherein the monitor comprises a microscope, coupled to the controller and the user interface, to allow observation of the cells on the display screen.

15. A method for preparing donor cells for a cell therapy, the method comprising:
disposing the donor cells in a culturing medium;
creating a cardiac electrical condition in the culturing medium, the cardiac electrical condition simulating an electrical condition of a myocardium;
creating a mechanical stress upon the donor cells, the mechanical stress simulating mechanical forces applied upon cardiac muscle cells in the myocardium; and
introducing one or more exogenous agents to the culturing medium to change one or more biological properties of the donor cells.

16. The method of claim 15, wherein creating the cardiac electrical condition comprises delivering pacing pulses to the donor cells.

17. The method of claim 16, wherein delivering the pacing pulses comprises delivering pacing pulses having a pacing voltage of 0.1 to 10 volts and a pacing pulse width of 0.1 to 10 milliseconds.

18. The method of claim 16, wherein delivering the pacing pulses comprises delivering the pacing pulses continuously for a predetermined duration.

19. The method of claim 18, wherein delivering the pacing pulses comprises delivering the pacing pulses continuously for 1 to 14 days.

20. The method of claim 16, wherein delivering the pacing pulses comprises delivering the pacing pulses for a predetermined duration interrupted by one or more non-pacing periods.

21. The method of claim 20, wherein delivering the pacing pulses comprises delivering the pacing pulses at a duty cycle of 5 to 75 percent for 1 to 14 days.

22. The method of claim 15, wherein creating the cardiac electrical condition comprises applying an electrical filed to the culturing medium.

23. The method of claim 22, wherein applying the electrical field comprises applying a static electrical filed having a strength of 1 to 100 volts per meter.

24. The method of claim 22, wherein applying the electrical field comprises applying the electrical field continuously for a predetermined duration.

25. The method of claim 24, wherein applying the electrical field comprises applying the electrical field continuously for 1 to 14 days.

26. The method of claim 22, wherein applying the electrical field comprises applying the electrical field for a predetermined duration interrupted by one or more non-stimulating periods.

27. The method of claim 26, wherein applying the electrical field comprises applying the electrical field at a duty cycle of 5 to 75 percent for 1 to 14 days.

28. The method of claim 15, wherein creating the mechanical stress comprises subjecting the donor cells to a cyclic mechanical force so that the donor cells are cyclically stretched and relaxed.

29. The method of claim 28, wherein creating the mechanical stress comprises extending the donor cells in at least one direction by approximately 5 to 20 percent of their length at a predetermined frequency of 0.25 to 2 hertz.

30. The method of claim 28, wherein creating the mechanical stress comprises applying the mechanical stress continuously for a predetermined duration.

31. The method of claim 30, wherein creating the mechanical stress comprises applying the mechanical stress continuously for 1 to 14 days.

32. The method of claim 28, wherein creating the mechanical stress comprises applying the mechanical stress for a predetermined duration interrupted by one or more non-stimulating periods.

33. The method of claim 32, wherein creating the mechanical stress comprises applying the mechanical stress at a duty cycle of 5 to 75 percent for 1 to 14 days.

34. The method of claim 15, wherein introducing the one or more exogenous agents comprises introducing one or more of
a differentiation factor;
a growth factor;
an angiogenic protein;
a survival factor;
a cytokine; and
an expression cassette (transgene) encoding a gene product including one or more of an angiogenic protein, a growth factor, a differentiation factor, a survival factor, a cytokine, a cardiac cell-specific structural gene product, a cardiac cell-specific transcription factor, a membrane protein, and an antisense sequence.

35. The method of claim 15, further comprising providing an image of the donors cells in the culturing module to allow observation by a user.

36. A method for treating damaged cardiac tissue, the method comprising:
conditioning donor cells in vitro;
injecting the conditioned donor cells into a predetermined region in a heart; and
delivering pacing pulses to the heart using an implantable pacemaker.

37. The method of claim 36, wherein conditioning the donor cells in vitro comprises applying one or more of an electrical stimulation, a mechanical stimulation, and a biological stimulation to the donor cells, wherein the donor cells are disposed in a culturing medium.

38. The method of claim 37, wherein applying the electrical stimulation comprises delivering pacing pulses to the donor cells.

39. The method of claim 38, wherein applying the electrical stimulation further comprise applying a static electrical field to the donor cells.

40. The method of claim 37, wherein applying the mechanical stimulation comprises cyclically deforming the donor cells.

41. The method of claim 40, wherein cyclically deforming the donor cells comprises stretching and relaxing the donor cells at a predetermined frequency.

42. The method of claim 37, wherein applying the biological stimulation comprises releasing chemical or biochemical agents to the culturing medium.

43. A system for electrical therapy of cardiac tissue of a heart, at least a portion of the cardiac tissue administered with exogenous cells in a cell therapy, the system comprising:
an atrial lead, a first ventricular lead, and a second ventricular lead each including one or more electrodes allowing for one or more of delivering electrical pulses and sensing electrical signals; and
a pulse generator including an interface for connections to the atrial lead, the first ventricular lead, and the second ventricular lead, a controller programmable for a plurality of pulse delivery modes, and a sense amplifier for sensing the electrical signals from the atrial lead, the first ventricular lead, and the second ventricular lead, wherein the pulse generator includes a selectable pacing mode for providing therapeutic electrical stimulation to enhance the cell therapy of the cardiac tissue.

44. The system of claim 43, wherein the therapeutic electrical stimulation includes a VDD pacing mode having an atrioventricular delay which is short compared to an intrinsic atrioventricular delay of the heart.

45. The system of claim 44, wherein the atrioventricular delay is varied gradually over time.

46. The system of claim 43, wherein the therapeutic electrical stimulation includes a DDD pacing mode having an atrioventricular delay which is short compared to an intrinsic atrioventricular delay of the heart.

47. The system of claim 46, wherein the atrioventricular delay is varied gradually over time.

48. The system of claim 43, wherein the therapeutic electrical stimulation includes a biventricular pacing mode having a first atrioventricular delay and a second atrioventricular delay.

49. The system of claim 43, wherein the therapeutic electrical stimulation includes a biventricular pacing mode having an atrioventricular delay and an interventricular delay.

50. A method for enhancing cell therapy of cardiac tissue, the method comprising:
applying a pacing therapy using an implantable pulse generator to cardiac tissue administered with exogenous cell therapy comprising donor cells,
wherein the electrical therapy enhances one or more of engraftment, survival, proliferation, differentiation or function of the donor cells.

51. The method of claim 50, wherein the pacing therapy includes DDD-mode pacing with an atrioventricular delay which is relatively short compared to an intrinsic atrioventricular interval.

52. The method of claim 51, wherein the atrioventricular delay is varied gradually over time.

53. The method of claim 50, wherein the pacing therapy includes biventricular pacing with a first atrioventricular delay and a second atrioventricular delay.

54. The method of claim 50, wherein the pacing therapy includes biventricular pacing with an atrioventricular delay and an interventricular delay.

55. The method of claim 50, wherein the donor cells are conditioned in vitro.

56. The method of claim 55, wherein the donor cells are electrically stimulated in vitro.

57. The method of claim 55, wherein the donor cells are mechanically stimulated in vitro.

58. The method of claim 55, wherein the donor cells are biologically stimulated in vitro.

59. The method of claim 50, wherein the pacing therapy is applied based on a level of activity.

60. The method of claim 59, wherein the pacing therapy is applied based on certain times of day.

61. The method of claim 50, wherein the pacing therapy is applied during periods of relative inactivity.

62. The method of claim 61, wherein the pacing therapy is applied during sleep.

63. The method of claim 50, wherein the pacing therapy is applied based on a level of stress.

64. A method comprising:
delivering an electrical energy to a mammal subjected to cell therapy, wherein:
the cell therapy includes administration of exogenous cells into a tissue; and
the electrical energy is delivered to the tissue hosting the exogenous cells to enhance one or more of engraftment, survival, proliferation and differentiation of the cells.

65. The method of claim 64, wherein delivering the electrical energy comprises delivering pacing pulses.

66. The method of claim 65, wherein delivering the pacing pulses comprises applying VDD-mode pacing with an atrioventricular delay which is relatively short compared to an intrinsic atrioventricular interval.

67. The method of claim 65, wherein delivering the pacing pulses comprises applying DDD-mode pacing with an atrioventricular delay which is relatively short compared to an intrinsic atrioventricular interval.

68. The method of claim 64, further comprising conditioning the exogenous cells in vitro.

69. The method of claim 68, wherein conditioning the exogenous cells in vitro comprises one or more of applying an electrical stimulation, a mechanical stimulation, and a biological stimulation.
